(19) United States
(12) Patent Application Publication
(10) Pub. No.: US 2026/0232988 A1
(43) Pub. Date: Aug. 13, 2026

(54) APPARATUS, SYSTEMS, AND METHODS FOR PERCUTANEOUS PNEUMATIC CARDIAC ASSISTANCE

(71) Applicant: PercAssist, Inc., Santa Clara, CA (US)

(72) Inventors: Albert K. CHIN, Santa Clara, CA (US); Gerardo NORIEGA, Santa Clara, CA (US); Sage MORELAND, Santa Clara, CA (US)

(21) Appl. No.: 19/638,371

(22) Filed: Apr. 3, 2026

Related U.S. Application Data
(63) Continuation of application No. PCT/US2024/050555, filed on Oct. 9, 2024.
(60) Provisional application No. 63/589,613, filed on Oct. 11, 2023.

(51) Int. Cl. A61M 60/289; A61M 39/02; A61M 60/187; A61M 60/497; A61M 60/515; A61M 60/569; A61M 60/861; A61M 60/865

(52) U.S. Cl. CPC A61M 60/289; A61M 39/02; A61M 60/187; A61M 60/497; A61M 60/515; A61M 60/569; A61M 60/861; A61M 60/865; A61M 2205/0216; A61M 2205/04; A61M 2205/15; A61M 2205/3303; A61M 2205/3327; A61M 2205/50; A61M 2210/122; A61M 2210/125; A61M 2230/005; A61M 2230/04

(57) ABSTRACT

Described herein are an implantable cardiac assist system and methods of delivery and use thereof. The system can include one or more of an external drive unit, an anchor wire, an implantable cardiac assist catheter, and a pneumatic effector. The pneumatic effector can be a balloon. The anchor wire can include a spiral anchor at the distal tip for anchoring the pneumatic. The system can be used with ECG electrodes to monitor the rhythm of the heart and adjust the action of the pneumatic effector based on the rhythm of the heart.

### CROSS-REFERENCE TO RELATED APPLICATIONS

The present application is a continuation of PCT Application No. PCT/US2024/050555, filed on Oct. 9, 2024, which claims priority to U.S. Provisional Patent Application No. 63/589,613, filed Oct. 11, 2023, the full disclosures of which are incorporated herein by reference.

The subject matter of the present disclosure is related to that of U.S. patent application Ser. No. 17/411,928, filed Aug. 25, 2021, now U.S. Pat. No. 12,097,364, which was issued on Sep. 24, 2024, and PCT Application No. PCT/US2023/067920 filed Jun. 5, 2023, the full disclosures of which are incorporated herein by reference.

### BACKGROUND

The present disclosure is related to systems, device, and methods for cardiac assistance.

In the United States, as many as 50,000 patients per year require heart transplantation due to end stage heart failure. Donor hearts can be only available for 2,000 to 2,500 patients per year. Many patients who can be unable to receive transplants can survive by receiving a ventricular assist device (VAD).

### SUMMARY

Disclosed herein is an implantable cardiac assist system. The implantable cardiac assist system can comprise an external drive unit. The implantable cardiac assist system can comprise an anchor wire configured to be anchored to a pericardium of a subject. The implantable cardiac assist system can comprise an implantable cardiac assist catheter comprising a distal section. The implantable cardiac assist system can comprise a pneumatic effector configured to be operatively coupled to the external drive unit. In some cases, the pneumatic effector is coupled to the distal section of the implantable cardiac assist catheter and configured to be implanted beneath a pericardial sac of the subject and over a myocardial surface overlying a left ventricle of the subject. In some cases, the distal section of the implantable cardiac assist catheter has a guidewire lumen with a first port proximal to the pneumatic effector and a second port distal to the pneumatic effector. In some cases, the implantable cardiac assist catheter is configured to be advanced over the anchor wire via the guidewire lumen.

Provided herein is a method for implanting a cardiac assist system. The method can comprise inserting a guidewire into an intrapericardial space anterior to a heart of a subject at an apical site. The method can comprise advancing a vascular sheath and a tapered dilator over the guidewire. The method can comprise advancing a pericardial anchor wire to a lateral aspect of a pericardium of the heart. The method can comprise anchoring the pericardial anchor wire to the lateral aspect of the pericardium. The method can comprise advancing an implantable cardiac assist catheter over the anchor wire via a guidewire lumen of the implantable cardiac assist catheter with a first port proximal to the pneumatic effector and a second port distal to the pneumatic effector, thereby positioning the implantable cardiac assist catheter anterior to the heart.

Described herein is a cardiac assist system. The cardiac assist system can comprise an external drive unit. The cardiac assist system can comprise an implantable cardiac assist catheter comprising a distal section having a guidewire lumen. The cardiac assist system can comprise a pneumatic effector configured to be operatively coupled to the external drive unit, wherein the pneumatic effector is coupled to the distal section of the implantable cardiac assist catheter. In some cases, the distal section of the implantable cardiac assist catheter has a first port proximal to the pneumatic effector and a second port distal to the pneumatic effector. In some cases, the pneumatic effector is configured to be implanted beneath a pericardial sac of the subject and over a myocardial surface overlying a left ventricle of the subject. In some cases, the pneumatic effector comprises a partially elastic balloon configured to inflate with a cushioning effect on a myocardium of the subject. The cardiac assist system can comprise an anchor wire. In some cases, the anchor wire is configured to anchor the partially elastic balloon to a position anterior to the heart.

Provided herein is a method for implanting a cardiac assist system. The method can comprise anchoring a pericardial anchor wire to a lateral aspect of a pericardium. The method can comprise advancing a guidewire lumen of an implantable cardiac assist catheter over the anchor wire into a position anterior to a heart. The method can comprise anchoring a pneumatic effector to the position anterior to the heart, the pneumatic effector being coupled to a distal end of the implantable cardiac assist catheter and configured to be implanted beneath a pericardial sac of the subject and over a myocardial surface overlying a left ventricle of the subject, wherein the pneumatic effector comprises a partially elastic balloon. In some cases, the partially elastic balloon is configured to inflate with a cushioning effect on a myocardium of the subject at the position anterior to the heart in response to gas being driven thereto.

Described herein is a method for assisting cardiac function in a subject suffering from heart failure. The method can comprise measuring the subject's ECG to determine a cardiac rhythm. The method can comprise delivering a driving gas to expand and contract a pneumatic effector implanted over the subject's left ventricle in response to the measured ECG. In some cases, the pneumatic effector comprises a partially elastic balloon. In some cases, the driving gas delivery is synchronized with the determined cardiac rhythm to cause the partially elastic balloon to compress a heart of the subject at a rate which matches the cardiac rhythm. In some cases, the partially elastic balloon is configured to inflate with a cushioning effect on a myocardium of the subject at a position anterior to the heart in response to gas being driven thereto.

Provided herein is a cardiac assist system. The cardiac assist system can comprise an external drive unit. The cardiac assist system can comprise an implantable cardiac assist catheter comprising a distal section. The cardiac assist system can comprise a pneumatic effector configured to be operatively coupled to the external drive unit. In some cases, the pneumatic effector is coupled to the distal section of the implantable cardiac assist catheter and configured to be implanted beneath a pericardial sac of the subject and over a myocardial surface overlying a left ventricle of the subject. In some cases, the pneumatic effector comprises a balloon having a size of at least about 120 cc and configured to displace a left ventricular wall by at least about 4 cm.

Described herein is a method for implanting a cardiac assist system. The method can comprise anchoring a pericardial anchor wire to a lateral aspect of a pericardium. The method can comprise advancing an implantable cardiac assist catheter into a position anterior to a heart. The method can comprise anchoring a pneumatic effector to the position anterior to the heart, the pneumatic effector being coupled to a distal end of the implantable cardiac assist catheter. In some cases, the pneumatic effector comprises a balloon having a size of at least about 120 cc. In some cases, the partially elastic balloon is configured to receive a driving gas delivered thereto to be inflated. In some cases, the partially elastic balloon is configured to displace a left ventricular wall of the heart by at least about 4 cm in response to being inflated.

Provided herein is a method for assisting cardiac function in a subject suffering from heart failure. The method can comprise measuring the subject's ECG to determine a cardiac rhythm. The method can comprise delivering a driving gas to expand and contract a pneumatic effector implanted over the subject's left ventricle in response to the measured ECG. In some cases, the pneumatic effector comprises a balloon having a size of at least about 120 cc. In some cases, the balloon is configured to displace a left ventricular wall by at least about 4 cm while expanding the balloon. In some cases, the driving gas delivery is synchronized with the determined cardiac rhythm to cause the balloon to compress a heart of the subject at a rate which matches the cardiac rhythm.

Described herein is an implantable cardiac assist system. The implantable cardiac assist system can comprise an external drive unit. The implantable cardiac assist system can comprise an anchor wire configured to be anchored to a pericardium of a subject. The implantable cardiac assist system can comprise implantable cardiac assist catheter comprising a distal section. The implantable cardiac assist system can comprise a pneumatic effector configured to be operatively coupled to the external drive unit. In some cases, the pneumatic effector is coupled to the distal section of the implantable cardiac assist catheter and configured to be implanted beneath a pericardial sac of the subject and over a myocardial surface overlying a left ventricle of the subject. In some cases, the implantable cardiac assist catheter is configured to be advanced over the anchor wire via the guidewire lumen. In some cases, (i) the pneumatic effector comprises a balloon having a size of at least about 120 cc, wherein the balloon is configured to displace a left ventricular wall by at least about 4 cm while expanding the balloon; (ii) the pneumatic effector comprises a partially elastic balloon configured to inflate with a cushioning effect on a myocardium of the subject at a position anterior to the heart in response to gas being driven thereto; and/or (iii) the distal section of the implantable cardiac assist catheter has a guidewire lumen with a first port proximal to the pneumatic effector and a second port distal to the pneumatic effector.

Provided herein is a method for implanting a cardiac assist system. The method can comprise anchoring a pericardial anchor wire to a lateral aspect of a pericardium. The method can comprise advancing a guidewire lumen of an implantable cardiac assist catheter over the anchor wire into a position anterior to a heart. The method can comprise anchoring a pneumatic effector to the position anterior to the heart, the pneumatic effector being coupled to a distal end of the implantable cardiac assist catheter and configured to be implanted beneath a pericardial sac of the subject and over a myocardial surface overlying a left ventricle of the subject. In some cases, (i) the pneumatic effector comprises a balloon having a size of at least about 120 cc, wherein the balloon is configured to displace a left ventricular wall by at least about 4 cm while expanding the balloon; (ii) the pneumatic effector comprises a partially elastic balloon configured to inflate with a cushioning effect on a myocardium of the subject at a position anterior to the heart in response to gas being driven thereto; and/or (iii) the distal section of the implantable cardiac assist catheter has a guidewire lumen with a first port proximal to the pneumatic effector and a second port distal to the pneumatic effector.

Additional aspects and advantages of the present disclosure can become readily apparent to those skilled in this art from the following detailed description, wherein only illustrative embodiments of the present disclosure are shown and described. As can be realized, the present disclosure is capable of other and different embodiments, and its several details can be capable of modifications in various obvious respects, all without departing from the disclosure. Accordingly, the drawings and description should be regarded as illustrative in nature, and not as restrictive.

### INCORPORATION BY REFERENCE

All publications, patents, and patent applications mentioned in this specification can be herein incorporated by reference to the same extent as if each individual publication, patent, or patent application was specifically and individually indicated to be incorporated by reference.

### BRIEF DESCRIPTION OF THE DRAWINGS

The novel features of the present disclosure can be set forth with particularity in the appended claims. A better understanding of the features and advantages of the present disclosure can be obtained by reference to the following detailed description that sets forth illustrative embodiments, in which the principles of the present disclosure can be utilized, and the accompanying drawings (also “Figure” and “FIG.” herein), of which:

FIG. 1 shows a perspective view of an example cardiac assist system in accordance with example embodiments described herein.

FIGS. 2A-2B illustrate exploded perspective side views of an example spiral pericardial anchor system in accordance with example embodiments described herein.

FIGS. 3A-3C depict perspective side views of an example distal end of the spiral pericardial anchor in accordance with example embodiments described herein.

FIGS. 4A-4B depict side views of an example partially slotted stainless steel anchor tube in accordance with example embodiments described herein.

FIGS. 5A-5C depict perspective side views of an alternate embodiment of a spiral pericardial anchor system in accordance with example embodiments described herein.

FIG. 6 illustrates a frontal section view of the position of the heart and the left lung, and the anatomical layers of pericardium, extra-pericardial fat, and pleura pertinent to an example spiral pericardial anchor placement in accordance with example embodiments described herein.

FIGS. 7A-7B depict magnified perspective side views of an example method of insertion of an example spiral anchor in accordance with example embodiments described herein.

FIG. 8 illustrates a transparent frontal view of an example method of vascular sheath placement into the pericardial sac over a previously placed guidewire in accordance with example embodiments described herein.

FIG. 9 shows a transparent frontal view of the advancement of an example spiral pericardial anchor system through a vascular sheath into the pericardial membrane in accordance with example embodiments described herein.

FIG. 10 shows a frontal view of the advancement of an example ventricular assist balloon cannula over an example spiral pericardial anchor embedded in the pericardial membrane in accordance with example embodiments described herein.

FIG. 11 shows a transparent frontal view of the attachment of the proximal end of an example spiral pericardial anchor to an example subcutaneous reservoir attached to an example ventricular assist balloon cannula in accordance with example embodiments described herein.

FIGS. 12A-12D illustrate schematic views of example configurations of a pump assembly suitable for use with an example cardiac assist systems in accordance with example embodiments described herein.

FIGS. 13A-13B illustrate top down (FIG. 13A) and side (FIG. 13B) views of a first example implantable port configuration in accordance with example embodiments described herein.

FIG. 14A-14B illustrate top down (FIG. 14A) and side (FIG. 14B) views of a second example implantable port embodiment in accordance with example embodiments described herein.

FIG. 15A-15B illustrate top down (FIG. 15A) and side (FIG. 15B) views of a third example implantable port configuration in accordance with example embodiments described herein.

FIG. 16 illustrates a perspective view of the interaction between an example cannula and an electrically conductive mesh embedded in an example implantable port in accordance with example embodiments described herein.

FIG. 17A illustrates a perspective view of a needle cannula having retaining bulbs in accordance with example embodiments described herein.

FIG. 17B illustrates a perspective view of a needle or other cannula in accordance with the present invention having retaining barbs in accordance with example embodiments described herein.

FIGS. 18A-18B illustrate a perspective (FIG. 18A) and frontal (FIG. 18B) view of an example alternative cannula in accordance with example embodiments described herein.

FIG. 19 illustrates a schematic side view of an implantable port suitable for use with the access cannula of FIGS. 18A and 18B in accordance with example embodiments described herein.

FIG. 20 illustrates a transparent perspective view of the cardiac assist system of FIG. 1 implanted in a patient in accordance with example embodiments described herein.

FIG. 21 shows a perspective view of a sectional view of the human torso depicting anatomy encountered during access to the pericardium and the heart in accordance with example embodiments described herein.

FIGS. 22A-22H depict perspective views of steps used during needle pericardial entry and percutaneous insertion of the cardiac assist system in accordance with example embodiments described herein.

FIGS. 23A-23B show side (FIG. 23A) and cross-sectional (FIG. 23B) views of the configuration of the catheter lumen in the cardiac assist system that accommodates the spiral pericardial anchor wire in accordance with example embodiments described herein.

FIG. 24 illustrates a front view of the anatomic structures observed during surgical access to the pericardial sac in accordance with example embodiments described herein.

FIGS. 25A-25E depict cross-sectional perspective views of steps used during open surgical pericardial access and insertion of the cardiac assist system into the pericardial sac in accordance with example embodiments described herein.

FIGS. 26A-26C illustrate frontal views of stabilization of the catheter body in a subcutaneous pocket prior to exit of the catheter body from the patient in accordance with example embodiments described herein.

FIG. 27 depicts a schematic of an example logic flow diagram illustrating operation of the external drive unit of an example cardiac assist system in accordance with example embodiments described herein.

### DETAILED DESCRIPTION

The present disclosure generally relates to medical devices, systems, and methods. More particularly, the present disclosure may relate to systems for providing cardiac assist to patients suffering from late-stage heart failure.

Ventricular assist devices (VADs) can be used in supporting blood circulation in patients suffering from severe heart failure. VADs can also be used to keep patients alive until a donor heart is available for transplantation as a “bridge to transplant.”

Placement of VADs, however, may include a large surgical incision, such as either a sternotomy or thoracotomy. It may be difficult for a patient with heart failure to undergo such a large surgical procedure. Minimally invasive VADs, such as intravascular rotary pumps and external compressive devices, may use a large transcutaneous “umbilical cord” to provide the power to drive the implanted pump assist component. Such cords can be inconvenient, uncomfortable, and present substantial risk of infection. Moreover, when infected, removal of the large cords may include surgical intervention.

For these reasons, it may be desirable to provide improved apparatus, systems and methods for providing cardiac assist devices to patients suffering from late-stage heart failure. It may be particularly desirable to provide such systems which can be implanted by subxiphoid and other minimally invasive routes. It may be further desirable to provide VAD systems having percutaneous connectors which reduce the risk of infection and which, if they become infected, can be easier to replace and disinfect. At least some of these features may be met by the disclosures described and claimed herein.

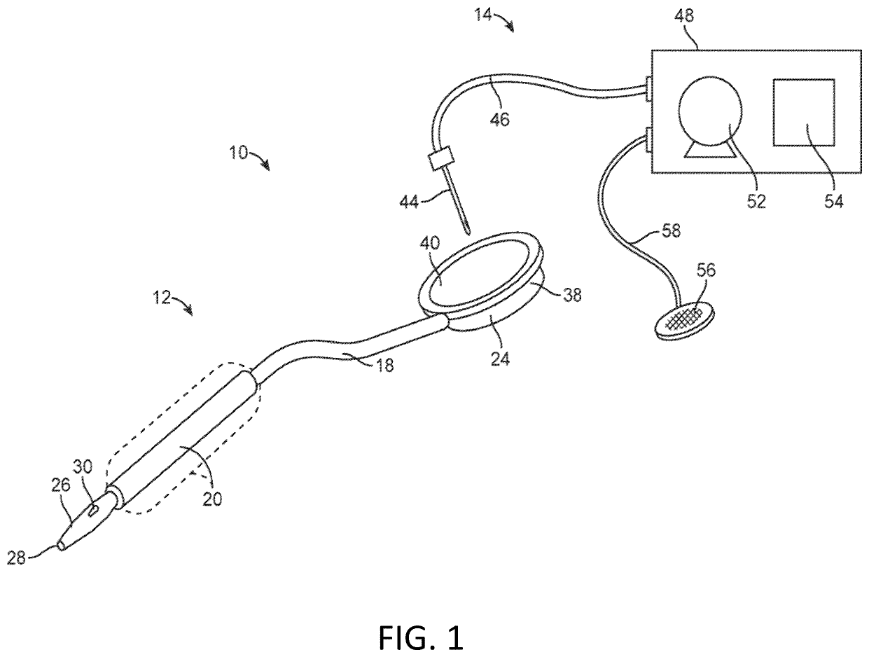

Disclosed herein is a minimally invasive cardiac assist device for patients suffering from heart failure or other compromised cardiac function. The device can comprise an implantable cardiac assist catheter. The catheter may be configured to be advanced over a guidewire to a position in the patient's pericardial sac between a myocardial surface and an inner surface of the pericardium. As shown in FIG. 1, The implantable cardiac assist catheter can have a balloon or other pneumatic effector at its distal end. An external drive unit can be provided to synchronize balloon inflation with ventricular contractions of the patient's natural cardiac cycle to compress the heart and provide ventricular assistance, thereby increasing the patient's cardiac output. The relative inflexibility of the fibrous pericardial sac can provide a restrictive constraint that causes the inflating balloon to compress the ventricle. In addition, the sternum also overlies the heart and can provide a bony enclosure to enhance cardiac compression upon balloon inflation. The cardiac assist device may be used with a sensor to obtain an electrocardiographic signal for synchronized balloon inflation.

### Anchor

Described herein can be anchors that may be used for anchoring a pneumatic effector in an implanted location.

In some cases, a balloon cannula may be inserted inside the pericardial sac and positioned anterior to the left ventricle of the heart. Inflation of the balloon during cardiac systole and deflation of the balloon during cardiac diastole may be conducted to increase cardiac output in patients with congestive heart failure. The ventricular assist balloon cannula may be inserted through the pericardium at the inferior aspect of the heart near the apex via a subxiphoid incision or needle puncture. The distal end of the balloon cannula may be advanced to the left lateral aspect of the heart immediately inferior to the left atrial appendage, resulting in positioning of the balloon anterior to the left ventricle. A fluid-tight reservoir may be attached to the proximal end of the balloon cannula, and the reservoir may be implanted subcutaneously in the subxiphoid region. The fluid-tight reservoir can comprise a balloon. Intrapericardial balloon inflation can be performed via a battery-operated air pump residing outside the body of the patient. A large bore needle may pierce through the patient's skin and the elastomeric sealing face of the subcutaneous reservoir, transmitting flow from the air supply line in the external unit to the intrapericardial balloon cannula.

Upon cyclical inflation and deflation of the balloon, it may be observed that the balloon cannula can migrate out of position over the left ventricle, and it can skew towards the right side of the heart. This may lead to a loss of left ventricular compression and ineffective left ventricular assistance. Therefore, it may be desirable to provide an anchoring system for the tip of the balloon cannula. It can be additionally desirable to provide an anchoring system for the ventricular assist balloon cannula that can allow exchange of the balloon cannula while preserving the favorable position established by the original anchor system. The balloon can have a finite life span; for example, less than about 6 months, about 6 months, about one year, about 2 years, or greater than about 2 years. Provision of an anchor system that allows balloon cannula exchange may simplify and shorten subsequent balloon replacement procedures.

A trans-pericardial anchor catheter to stabilize the distal end of an intrapericardial balloon cannula can comprise a small diameter non-collapsible catheter body with a short distal section comprised of a braided sheath formed of multiple polymer strands. A rounded tip may be attached to the distal end of the braided sheath, and a stainless-steel wire may be attached to the tip extending the length of the braided sheath and the catheter body. A length of stainless-steel tubing may be bonded to the proximal portion of the catheter body, and the stainless-steel tubing can extend approximately one centimeter proximal to the proximal end of the catheter. The steel wire inside the catheter may be a slip fit with the inner diameter of the stainless-steel tube, and it may protrude several centimeters proximal to the proximal end of the stainless-steel tube. Traction on the stainless-steel wire while the catheter is held stationary can cause the braided sheath to form an expanded disc. The braided sheath may be maintained in its expanded configuration by crimping the stainless-steel tube onto the inner stainless-steel wire.

The pericardial anchor catheter may function well as a balloon cannula stabilizing device. However, its placement technique can be hazardous to the patient, as the entire length of the braided sheath may exit out of the pericardial sac prior to expansion of the braided sheath to form the anchoring disc. The patient's lung can be close to the pericardial sac, separated from the pericardium by a few millimeters of extra-pericardial fat and a pleural membrane that is normally less than half of a millimeter thick. The braided sheath length may be greater than 10 mm; therefore, anchor placement may cause perforation of the surface of the lung.

A pericardial anchor is proposed that can involve an elongated, small-bore stainless-steel tube attached to the center of a distal unit comprised of a disc containing a flat distal surface with a spiral rigid coil circumferentially attached to the disc such that a single revolution of the spiral coil extends distal to the flat surface of the disc. The distal portion of the spiral coil may lie in a plane orthogonal to the axis of the tube, and the tip of the spiral may contain an angled undercut, to form a point for pericardial entry. A removeable handle can be provided at the proximal end of the stainless-steel tube. The removeable handle may be a guidewire torquing device, comprised of a short plastic body pin vise, that is clamped onto the stainless-steel tube.

The spiral pericardial anchor system may be inserted into the pericardial sac via an opening formed near the apical aspect of the heart. The opening may be an incision of the pericardium performed via a surgical pericardial window procedure. Alternatively, access into the pericardial sac may be performed percutaneously via needle entry through the skin in the subxiphoid region, advancement of the needle through the pericardium, insertion of a guidewire through the needle, removal of the needle, and advancement of a vascular sheath containing an inner tapered dilator over the guidewire. Upon removal of the tapered dilator and guidewire, the spiral pericardial anchor may be advanced through the vascular sheath into the intrapericardial space. In some cases, the physician may wish to leave the guidewire in position upon removal of the tapered dilator and advance the spiral pericardial anchor system over the guidewire, to guide the anchor system to the desired pericardial location. The pericardial anchor system may employ a stainless-steel tube with an inner lumen to accommodate guidewire placement, rather than a solid stainless-steel wire or rod. Once the pericardial anchor system has been advanced to the desired anchor site, the guidewire may be removed from the lumen of the device, and the spiral tip of the device may be pressed against the pericardium with a constant force of approximately one pound. The spiral may be rotated using the removeable handle multiple revolutions until resistance is met. In some cases, at this point, one revolution of the spiral anchor has exited the pericardium, and the pericardial membrane has wedged in the apex formed by the flat distal face of the disc and the exposed spiral coil. Resistance felt upon gentle retraction of the anchor system can indicate that proper placement has been achieved.

The elongated stainless-steel tube extending proximally from the spiral coil and the supporting disc may contain a series of radially offset microscopic slots extending one-half of the distal length and the full thickness of the tube. The narrow slots can be approximately 0.002″ wide, and they may be formed in the stainless-steel tube using a laser cutter. These slots can impart multi-directional flexibility to the portion of the tube in contact with the heart inside the pericardial sac, to avoid potential myocardial trauma upon long term implantation of the spiral anchor. Adjacent rows of circumferential microscopic slots may be offset with respect to the previous row, imparting axial flexibility while retaining the column strength and torsional stiffness of the tube required for exertion of a one-pound normal force against the pericardium while rotating the spiral to enable its pointed tip to enter the pericardial membrane. Adjacent slots can be spaced a uniform distance for much of the length of the slotted tube. The proximal and distal slotted sections may feature adjacent slots that increase linearly in distance as they proceed away from the center of the slotted portion of the tube. Increased distance between slots can provide a strain relief at the point at which the rigid tube becomes flexible, to avoid kinking of the tube at the junction between the solid and slotted sections. The proximal portion of the stainless-steel tube may contain a solid wall, as it often must be rigidly attached to the reservoir of the balloon cannula via a setscrew that compresses and deforms the tube during attachment. A solid proximal section of stainless-steel tube may be required at the reservoir attachment point to ensure proper balloon cannula anchoring, and to avoid any potential of anchor tube fracture over the life of the implant.

An alternate embodiment of the device may employ a stainless-steel tube with an off-round cross section, and a long co-axial slip-fit outer polymer sleeve with a similar cross-sectional profile which is gripped to deploy the spiral anchor. The cross-sectional profile of the stainless-steel tube and the corresponding cross-sectional profile of the outer sleeve keyed to the inner sleeve may be oval, square, hexagonal, or other off-round shape. The increased profile of the outer sleeve can enable it to be used as the grip during spiral anchor placement. A flexible cap may be placed on the proximal end of the stainless-steel tube to stabilize the outer polymer sleeve during spiral anchor placement. Following spiral anchor placement, the proximal end cap may be slipped off the stainless-steel tube, and the outer polymer sleeve removed. This embodiment may not require the use and the removal of the torquer handle component. Torquer grip removal may result in the physician exerting a twisting motion of both hands, which may dislodge the spiral anchor from the pericardium. In some embodiments, the flexible cap is pulled axially with the outer sleeve held stationary, thus avoiding most torsional movement that may dislodge the spiral anchor.

The spiral anchor may place one revolution of the spiral through the pericardium. The wire diameter of the spiral may be approximately 0.022″ (0.56 mm), and one revolution of the spiral may have a depth of approximately 0.056″ (1.4 mm). In some cases, the wire diameter of the spiral may be from about 0.015″ to 0.03″. In some cases, the wire diameter of the spiral may be from about 0.015″ to about 0.018″, about 0.015″ to about 0.021″, about 0.015″ to about 0.024″, about 0.015″ to about 0.027″, about 0.015″ to about 0.03″, about 0.018″ to about 0.021″, about 0.018″ to about 0.024″, about 0.018″ to about 0.027″, about 0.018″ to about 0.03″, about 0.021″ to about 0.024″, about 0.021″ to about 0.027″, about 0.021″ to about 0.03″, about 0.024″ to about 0.027″, about 0.024″ to about 0.03″, or from about 0.027″ to about 0.03″. In some cases, the wire diameter of the spiral may be less than about 0.015″, less than about 0.018″, less than about 0.021″, less than about 0.024″, less than about 0.027″, or less than about 0.03″. In some cases, the wire diameter of the spiral may be greater than about 0.015″, greater than about 0.018″, greater than about 0.021″, greater than about 0.024″, greater than about 0.027″, or greater than about 0.03″.

In some cases, one revolution of the spiral may have a depth from about 0.05″ to about 0.06″. In some cases, one revolution of the spiral may have a depth from about 0.05″ to about 0.052″, about 0.05″ to about 0.054″, about 0.05″ to about 0.056″, about 0.05″ to about 0.058″, about 0.05″ to about 0.06″, about 0.052″ to about 0.054″, about 0.052″ to about 0.056″, about 0.052″ to about 0.058″, about 0.052″ to about 0.06″, about 0.054″ to about 0.056″, about 0.054″ to about 0.058″, about 0.054″ to about 0.06″, about 0.056″ to about 0.058″, about 0.056″ to about 0.06″, or from about 0.058″ to about 0.06″. In some cases, one revolution of the spiral may have a depth less than about 0.05″, less than about 0.052″, less than about 0.054″, less than about 0.056″, less than about 0.058″, or less than about 0.06″. In some cases, one revolution of the spiral may have a depth greater than about 0.05″, greater than about 0.052″, greater than about 0.054″, greater than about 0.056″, greater than about 0.058″, or greater than about 0.06″.

The mean thickness of the epicardial fat layer in normal individuals without coronary artery disease is 4.4±1.2 mm. Therefore, one revolution of spiral at 1.4 mm may not protrude through the epicardial fat layer, and it may stop short of the pleural membrane that surrounds the lung. Hence, lung injury may not occur upon insertion of the spiral anchor into the pericardium.

After placement of the spiral anchor, the ventricular assist balloon cannula can be advanced along the stainless-steel tube component of the anchor into position inside the pericardial sac. An open through-lumen may extend the full length of the balloon, and this through-lumen may accommodate the stainless-steel anchor tube. Following placement of the balloon cannula into position, an implantable reservoir can be attached to the proximal end of the cannula. The proximal end of the stainless-steel anchor tube may be inserted into a channel on the side of the reservoir housing, and a setscrew that extends into the channel may be tightened onto the stainless-steel anchor tube to secure the balloon cannula in position throughout the duration of implantation.

Provided herein can be pericardial anchors for positioning a cardiac assist system over a patient's heart beneath the patient's sternum and ribs. An exemplary anchor may comprise a tubular shaft having a guidewire lumen therethrough and a pericardial anchor at a distal end of the tubular shaft. The pericardial anchor may be configured to anchor in the patient's pericardium in response to manipulation of the tubular shaft.

In some embodiments, the pericardial anchor further comprises a removable handle at a proximal end of the tubular shaft. The pericardial anchor may be configured to anchor in the patient's pericardium in response to manipulation of the tubular shaft via the handle. The tubular shaft may be configured to receive and anchor the cardiac assist system over the patient's heart beneath the patient's sternum and ribs after the handle is removed.

In some embodiments, the pericardial anchor further comprises an extension shaft configured to removably couple to the proximal end of the tubular shaft. The extension shaft may have a guidewire lumen. The guidewire lumen of the extension shaft may be co-axial with the guidewire or inner lumen of the tubular shaft. The distal end of the extension shaft may be removably fastened onto the proximal end of the tubular shaft. In some embodiments, the tubular shaft is a slotted metal tube having controlled flexibility. In some embodiments, manipulation of the tubular shaft comprises rotation of the tubular shaft.

In some embodiments, the pericardial anchor is a helical anchor with a flat distal face oriented in a plane orthogonal to an axis of the tubular shaft. The helical anchor may have a sharpened tip configured to penetrate a pericardial membrane with limited penetration into an underlying fat pad. In some embodiments, the pericardial anchor further comprises a polymer sleeve configured to cover the tubular shaft.

Described herein can be methods for positioning a cardiac assist system over a patient's heart beneath the patient's sternum and ribs. An exemplary method may comprise steps of: percutaneously advancing a guidewire to a position over the patient's heart beneath the patient's ribs; advancing a pericardial anchor at a distal end of a tubular shaft over the guidewire to position the pericardial anchor adjacent a preselected location on the patient's pericardium; implanting the pericardial anchor in the pericardium to stabilize the tubular shaft over the patient's heart; and advancing the cardiac assist system over the tubular shaft to locate the cardiac assist system over the patient's heart beneath the patient's sternum and ribs.

In some embodiments, the step of implanting the pericardial anchor in the pericardium comprises rotating the shaft to implant a helical anchor in the pericardium. The helical anchor may have a flat distal face oriented in a plane orthogonal to an axis of the tubular shaft. The helical anchor may have a sharpened tip configured to penetrate a pericardial membrane with limited penetration into an underlying fat pad.

In some embodiments, the cardiac assist system comprises a pneumatic effector configured to be implanted beneath a patient's pericardial sac and over a myocardial surface overlying the patient's left ventricle. In some embodiments, the cardiac assist system comprises an implantable port configured to receive a percutaneously introduced cannula, wherein said port is connected to supply a driving gas received from the cannula to the pneumatic effector. In some embodiments, the cardiac assist system comprises an external drive unit including a pump assembly and control circuitry configured to operate the pump to actuate the pneumatic effector in response to the patient's sensed heart rhythm. In some embodiments, the cardiac assist system comprises a connecting tube having a pump end attachable to the pump assembly and a cannula end attached to the cannula.

In some embodiments, after the cardiac assist system has been advanced over the tubular shaft to locate the cardiac assist system over the patient's heart beneath the patient's sternum and ribs, the cardiac assist system is locked in position with respect to the tubular shaft to prevent one or more of axial or transverse movement of the cardiac assist system with respect to the heart.

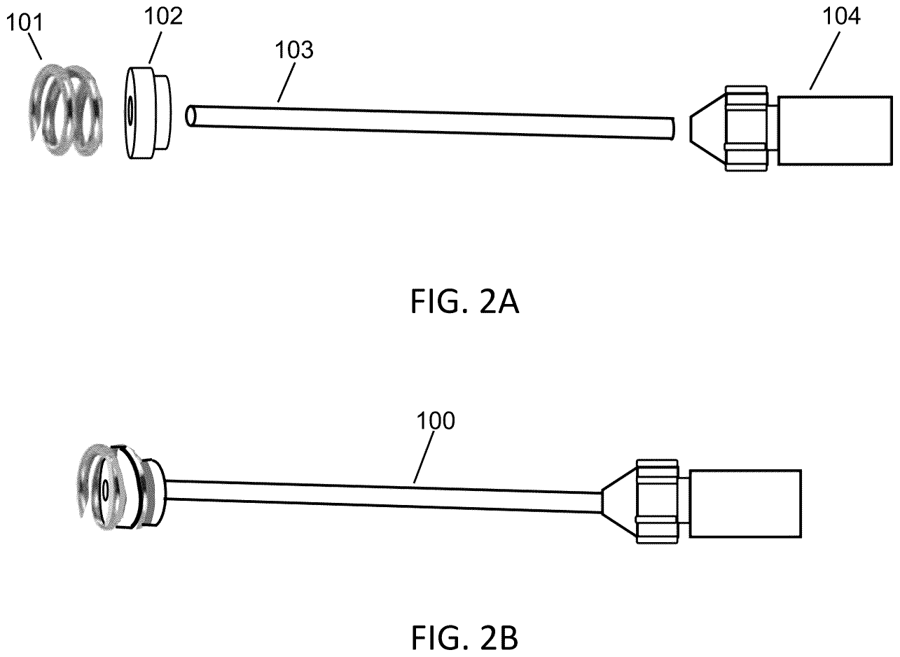

FIG. 2A depicts an exploded view of the components that form the spiral pericardial anchor system 100 as shown assembled in FIG. 2B. FIG. 2A shows a stainless-steel spiral form 101 with a pointed distal tip may be welded to a stainless-steel bushing 102, a long stainless-steel tube 103 may be attached to the center of the bushing 102, and guidewire torque device 104. Spiral structure 101 may be formed of stainless steel, with a wire diameter of approximately 0.022″ and an outer diameter measuring approximately 0.180″. The stainless-steel tube 103 may have an outer diameter of about 0.050″ and a wall thickness of about 0.005″, such that its inner lumen may accommodate an about 0.038″ guidewire. In some cases, the lumen may be larger so as to accommodate a larger guidewire. In some cases, the lumen may be smaller so as to better accommodate a smaller guidewire. A guidewire torque device 104 may be fastened onto the proximal end of the stainless-steel tube 103 to serve as a handle to facilitate rotation of the spiral pericardial anchor 100 during its placement in the pericardium. The guidewire torque device 104 is shown as a graspable block in FIGS. 2A and 2B. Alternatively or in combination, the proximal end of the stainless-steel tube 103 may be fastened onto the distal end of another stainless-steel tube so as to elongate or extend the stainless-steel tube 103. In some cases, the additional stainless-steel tube may be used as the guidewire torque device 104. In some cases, a separate guidewire torque device 104 may be fastened on to the proximal end of the additional stainless-steel tube. The additional stainless-steel tube may have an inner guidewire lumen configured to be coaxial with the inner lumen of the stainless-steel tube 103 as well.

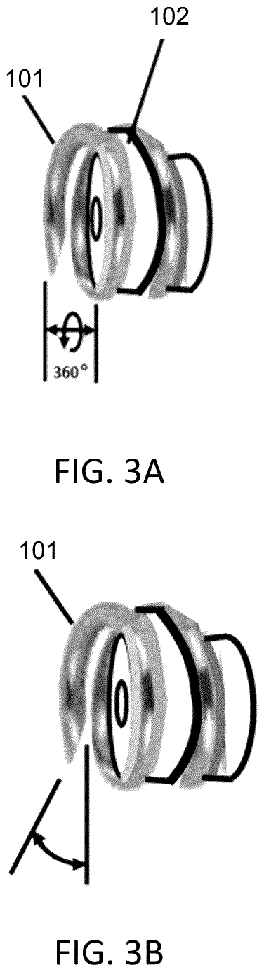

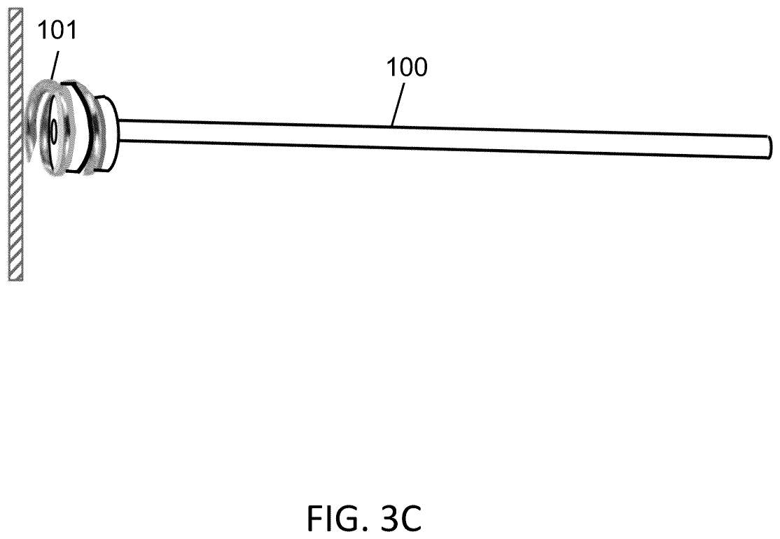

FIG. 3A illustrates the configuration of the spiral 101 as it is attached to the bushing 102. A single revolution of spiral 101 can extend distal to the flat distal face of bushing 102. FIG. 3B shows the configuration of the pointed distal tip of spiral 101, formed by grinding an angle on the inner aspect of the wire tip. An angled undercut of the wire may be used to form the distal tip, such that the distal portion of spiral 101 lies in a flat plane orthogonal to the axis of the spiral anchor system 100, as illustrated in FIG. 3C.

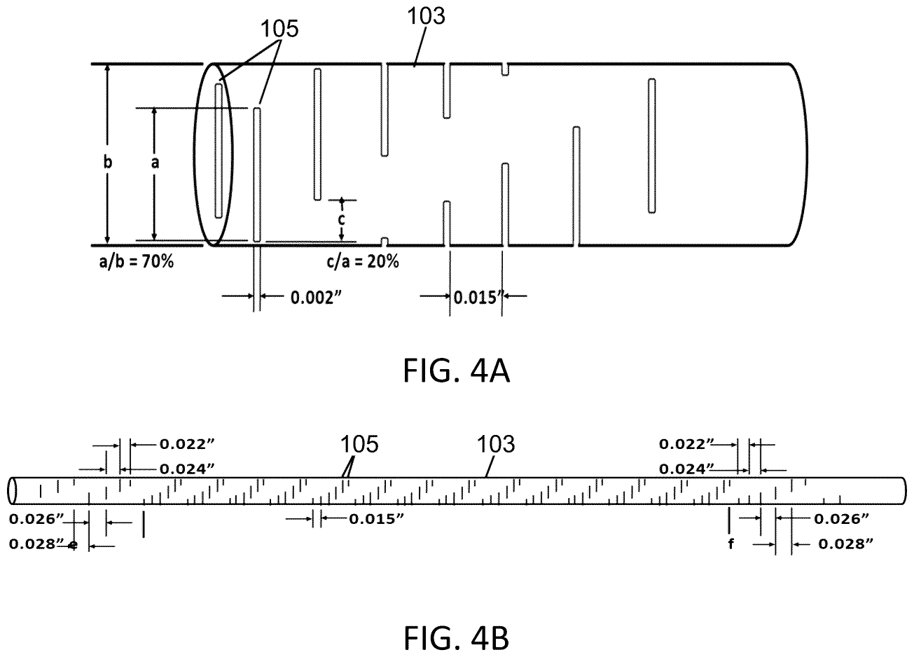

FIGS. 4A-4B depict the configuration of the stainless-steel tube 103, with microscopic slots 105 formed on opposing sides of its wall. The slots 105 may be invisible to the naked eye, at about 0.002″ in width, with a length that extends about 70% of the about 0.050″ diameter of the stainless-steel tube 103, equal to about 0.035″. In some cases, slots 105 may be from about 0.0015″ to 0.0025″ in width, with a length that extends from about 50% to about 90% of the from about 0.04″ to 0.06″ diameter of the stainless-steel tube 103, equal to from about 0.02″ to about 0.054″.

In some cases, the slots 105 may have a width from about 0.0015″ to about 0.0017″, about 0.0015″ to about 0.0019″, about 0.0015″ to about 0.0021″, about 0.0015″ to about 0.0023″, about 0.0015″ to about 0.0025″, about 0.0017″ to about 0.0019″, about 0.0017″ to about 0.0021″, about 0.0017″ to about 0.0023″, or from about 0.0017″ to about 0.0025″, about 0.0019″ to about 0.0021″, about 0.0019″ to about 0.0023″, about 0.0019″ to about 0.0025″, about 0.0021″ to about 0.0023″, about 0.0021″ to about 0.0025″, or from about 0.0023″ to about 0.0025″. In some cases, the slots 105 may have a width less than about 0.0015″, less than about 0.0017″, less than about 0.0019″, less than about 0.0021″, less than about 0.0023″, or less than about 0.0025″. In some cases, the slots 105 may have a width greater than about 0.0015″, greater than about 0.0017″, greater than about 0.0019″, greater than about 0.0021″, greater than about 0.0023″, or greater than about 0.0025″.

In some cases, slots 105 may comprise a length that extends from about 50% to about 90% of the diameter of the stainless-steel tube 103. In some cases, slots 105 may comprise a length that extends from about 50% to about 58%, about 50% to about 66%, about 50% to about 74%, about 50% to about 82%, about 50% to about 90%, about 58% to about 66%, about 58% to about 74%, about 58% to about 82%, about 58% to about 90%, about 66% to about 74%, about 66% to about 82%, about 66% to about 90%, about 74% to about 82%, about 74% to about 90%, or from about 82% to about 90% of the diameter of the stainless steel tube 103. In some cases, slots 105 may comprise a length that extends less than about 50%, less than about 58%, less than about 66%, less than about 74%, less than about 82%, or less than about 90% of the diameter of the stainless-steel tube 103. In some cases, slots 105 may comprise a length that extends greater than about 50%, greater than about 58%, greater than about 66%, greater than about 74%, greater than about 82%, or greater than about 90% of the diameter of the stainless-steel tube 103.

In some cases, the diameter of the stainless steel tube 103 is from about 0.04″ to about 0.044″, about 0.04″ to about 0.048″, about 0.04″ to about 0.052″, about 0.04″ to about 0.056″, about 0.04″ to about 0.06″, about 0.044″ to about 0.048″, about 0.044″ to about 0.052″, about 0.044″ to about 0.056″, about 0.044″ to about 0.06″, about 0.048″ to about 0.052″, about 0.048″ to about 0.056″, about 0.048″ to about 0.06″, about 0.052″ to about 0.056″, about 0.052″ to about 0.06″, or from about 0.056″ to about 0.06″. In some cases, the diameter of the stainless-steel tube 103 is less than about 0.04″, less than about 0.044″, less than about 0.048″, less than about 0.052″, less than about 0.056″, or less than about 0.06″. In some cases, the diameter of the stainless-steel tube 103 is greater than about 0.04″, greater than about 0.044″, greater than about 0.048″, greater than about 0.052″, greater than about 0.056″, or greater than about 0.06″.

In some cases, slots 105 may have a length from about 0.02″ to about 0.03″, about 0.02″ to about 0.04″, about 0.02″ to about 0.05″, about 0.02″ to about 0.054″, about 0.03″ to about 0.04″, about 0.03″ to about 0.05″, about 0.03″ to about 0.054″, about 0.04″ to about 0.05″, about 0.04″ to about 0.054″, or from about 0.05″ to about 0.054″. In some cases, slots 105 may have a length less than about 0.02″, less than about 0.03″, less than about 0.04″, less than about 0.05″, or less than about 0.054″. In some cases, slots 105 may have a length greater than about 0.02″, greater than about 0.03″, greater than about 0.04″, greater than about 0.05″, or greater than about 0.054″.

A distance of about 0.015″ can separate adjacent axial slots 105. A distance of from about 0.01″ to 0.02″ can separate adjacent axial slots 105. A distance of from about 0.01″ to about 0.012″, about 0.01″ to about 0.014″, about 0.01″ to about 0.016″, about 0.01″ to about 0.018″, about 0.01″ to about 0.02″, about 0.012″ to about 0.014″, about 0.012″ to about 0.016″, about 0.012″ to about 0.018″, about 0.012″ to about 0.02″, about 0.014″ to about 0.016″, about 0.014″ to about 0.018″, about 0.014″ to about 0.02″, about 0.016″ to about 0.018″, about 0.016″ to about 0.02″, or about 0.018″ to about 0.02″ can separate adjacent axial slots 105. A distance of less than about 0.01″, less than about 0.012″, less than about 0.014″, less than about 0.016″, less than about 0.018″, or less than about 0.02″ can separate adjacent axial slots 105. A distance of greater than about 0.01″, greater than about 0.012″, greater than about 0.014″, greater than about 0.016″, greater than about 0.018″, or greater than about 0.02″ can separate adjacent axial slots 105.

Adjacent slots 105 can be radially offset from the previous set of slots 105 by a distance equal to about 20% of the length of the slot 105, or about 0.035″. Adjacent slots 105 can be radially offset from the previous set of slots 105 by from about 15% to about 25% of the length of the slot 105. Adjacent slots 105 can be radially offset from the previous set of slots 105 by from about 15% to about 17%, about 15% to about 19%, about 15% to about 21%, about 15% to about 23%, about 15% to about 25%, about 17% to about 19%, about 17% to about 21%, about 17% to about 23%, about 17% to about 25%, about 19% to about 21%, about 19% to about 23%, about 19% to about 25%, about 21% to about 23%, about 21% to about 25%, or about 23% to about 25%. Adjacent slots 105 can be radially offset from the previous set of slots 105 by less than about 15%, less than about 17%, less than about 19%, less than about 21%, less than about 23%, or less than about 25%. Adjacent slots 105 can be radially offset from the previous set of slots 105 by greater than about 15%, greater than about 17%, greater than about 19%, greater than about 21%, greater than about 23%, or greater than about 25%.

Adjacent slots 105 can be radially offset from the previous set of slots 105 by from about 0.03″ to 0.04″. Adjacent slots 105 can be radially offset from the previous set of slots 105 by from about 0.03″ to about 0.032″, about 0.03″ to about 0.034″, about 0.03″ to about 0.036″, about 0.03″ to about 0.038″, about 0.03″ to about 0.04″, about 0.032″ to about 0.034″, about 0.032″ to about 0.036″, about 0.032″ to about 0.038″, about 0.032″ to about 0.04″, about 0.034″ to about 0.036″, about 0.034″ to about 0.038″, about 0.034″ to about 0.04″, about 0.036″ to about 0.038″, about 0.036″ to about 0.04″, or about 0.038″ to about 0.04″. Adjacent slots 105 can be radially offset from the previous set of slots 105 by less than about 0.03″, less than about 0.032″, less than about 0.034″, less than about 0.036″, less than about 0.038″, or less than about 0.04″. Adjacent slots 105 can be radially offset from the previous set of slots 105 by greater than about 0.03″, greater than about 0.032″, greater than about 0.034″, greater than about 0.036″, greater than about 0.038″, or greater than about 0.04″.

The slots 105 may extend approximately one half of the distal length of stainless-steel tube 103. They may impart flexibility to the portion of the stainless-steel tube 103 that lies inside the pericardial sac in contact with the heart. This flexibility may be essential in avoiding trauma to the heart during insertion of the spiral pericardial anchor and upon long term implantation. Excessive rigidity of the stainless-steel tube 103 may cause laceration or perforation of the heart during spiral anchor insertion, as well as potential myocardial laceration during long term implantation. The radially offset series of slots 105 can provide flexibility of the distal stainless-steel tube 103 in all directions without sacrificing the column strength or torsional strength required as the pericardial anchor exerts approximately one pound of force against the pericardium followed by its rotation to achieve pericardial entry and proper anchoring.

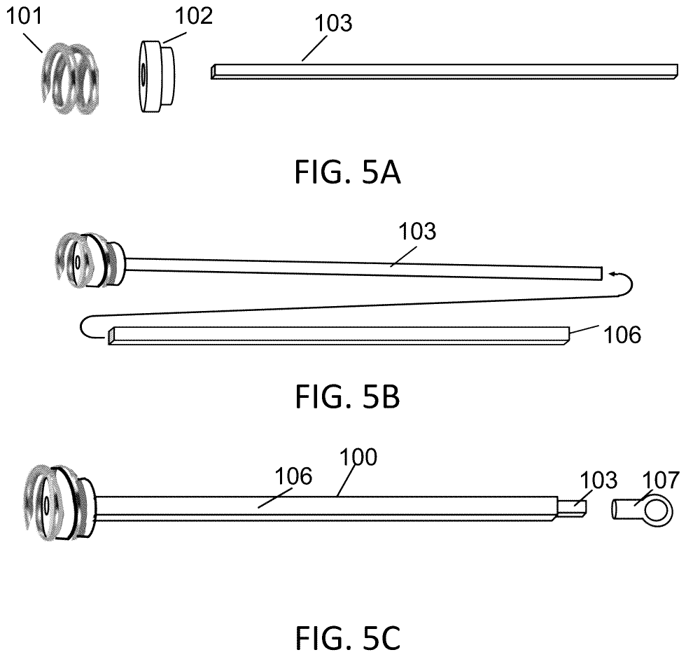

FIG. 5A depicts the inner components of an alternate embodiment of a spiral pericardial anchor system 100, comprising a spiral 101, a bushing 102, and a stainless-steel tube 103. In some cases, the stainless-steel tube 103 may contain an off-round cross-sectional profile such as a square. FIG. 5B shows that a telescoping, slip-fit polymer sleeve 16 may fit over off-round stainless-steel tube 103. The polymer sleeve 106 may contain an inner lumen that matches the outer profile of the off-round stainless-steel tube 103, and it may be slightly shorter in length than stainless steel tube 103. FIG. 5C shows that a flexible end cap 107 may be placed on the exposed proximal end of stainless-steel tube 103 to stabilize polymer sleeve 106 as it is grasped and rotated to insert the anchor system 100 in the pericardium. The proximal portion of polymer sleeve 106 may function as the handle for spiral anchor 101 insertion. Following anchor 101 insertion, the polymer sleeve 106 can be held stationary as the end cap 107 is removed from the stainless-steel tube 103. This may not require a twisting motion for removal of an attached torque device 104 as in the embodiment shown in FIG. 2B. Two-handed twisting required for removal of torque device 104 may dislodge the spiral 101 from the pericardium. Removal of end cap 107 can involve an axial motion that is less likely to dislodge a fixated spiral 101.

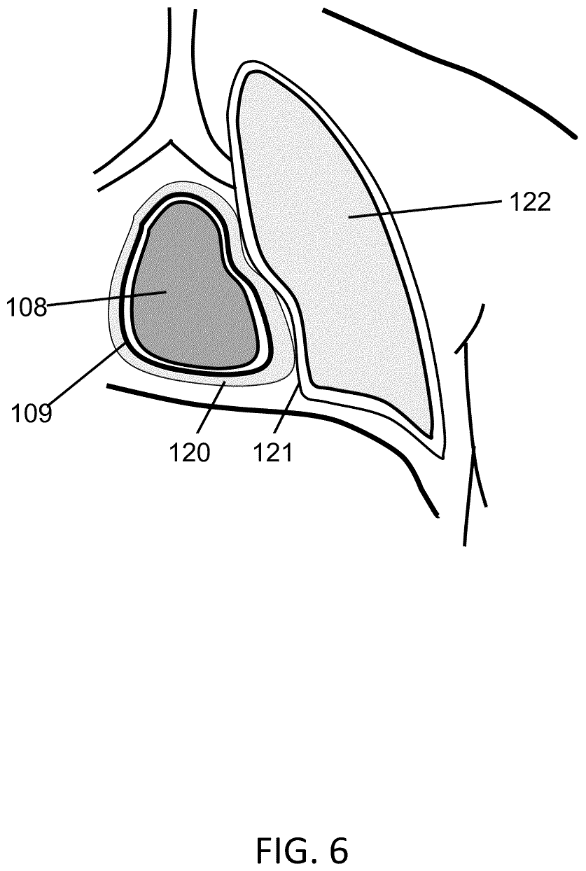

FIG. 6 depicts the heart 108 and left lung 122 in the thoracic cavity. The heart 108 can be enclosed by the pericardial sac 109. A pericardial fat pad 120 can lie outside of the pericardium 109, and a fibrous pleural membrane 121 can enclose the lung 122. The pleura 121 can be in contact with the pericardial fat pad 120.

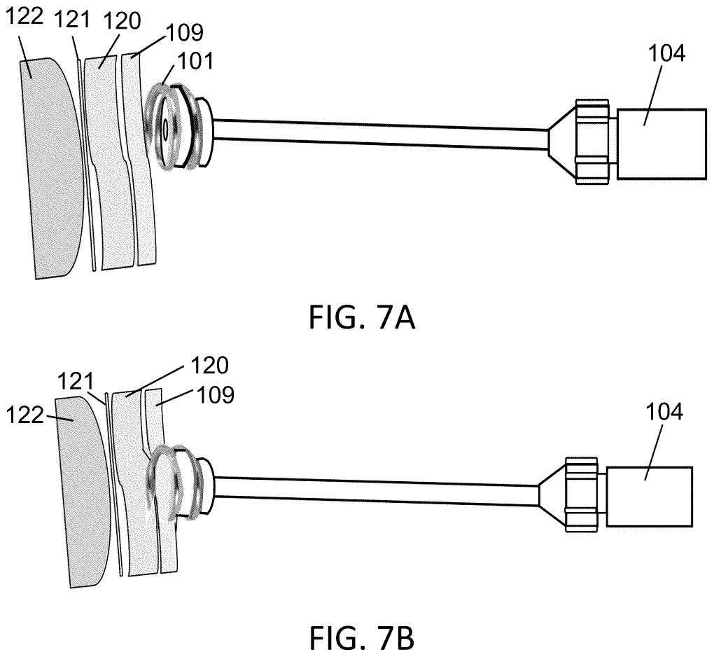

FIG. 7A shows the positioning of the spiral 101 and torque handle 104 in preparation for insertion into pericardial membrane 109. The extra-pericardial fat pad 120, pleura 121 and lung 122 can lie outside of pericardial membrane 109. The spiral 101 may be advanced to be in contact with the pericardial membrane 109 with a mild amount of force, approximately 1 pound of force, and rotated two to three revolutions using the torque handle 104 until resistance is felt, indicating that one revolution of spiral 101 has entered pericardial membrane 109 and pericardial membrane 109 is abutted against the distal face of bushing 102, as seen in FIG. 7B. Human pericardium has a mean thickness of about 1.02 mm, and the pericardial fat pad is approximately 4.4 mm thick. Therefore, the tip of spiral 101 upon placement may not enter the pleura 121 or the lung 22, avoiding potential for perforation or laceration of the lung 122.

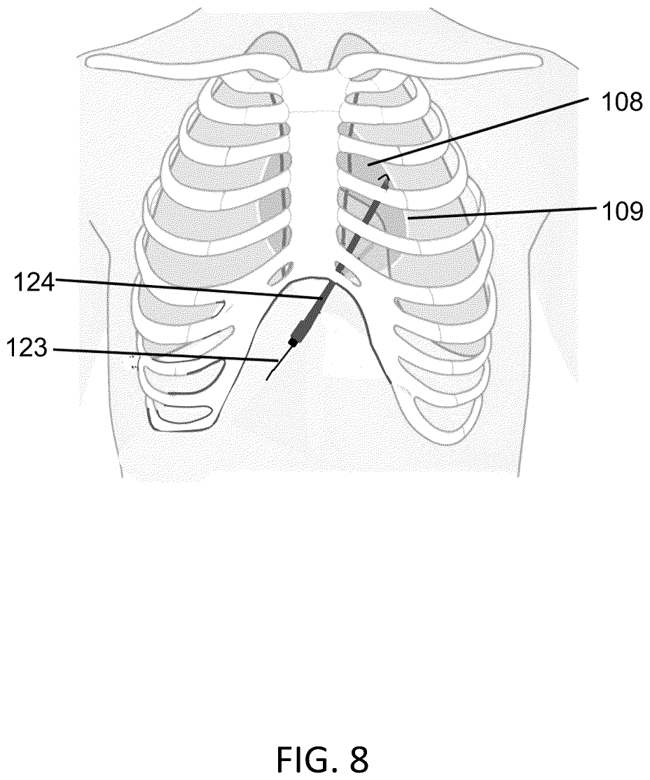

FIG. 8 depicts the positioning of vascular sheath 124 inside the pericardial sac 109 on the left lateral aspect of the heart 108, in preparation for placement of the spiral anchor catheter. Vascular sheath 124 may be advanced over a previously placed guidewire 123 inserted via a needle puncture in the pericardium 109 on the inferior aspect of the heart 108. Positioning of the guidewire 123 and the vascular sheath 124 may be performed under fluoroscopic x-ray guidance.

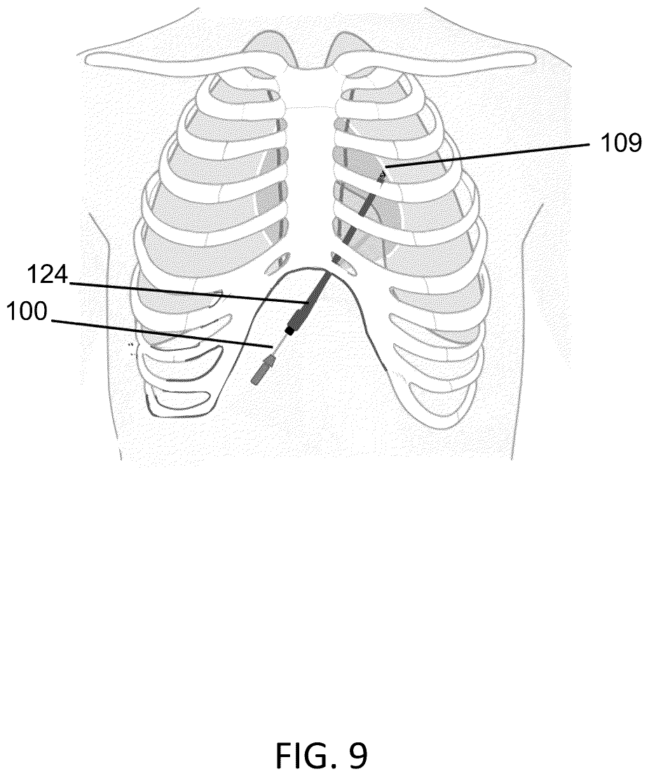

FIG. 9 shows advancement of the spiral anchor catheter 100 through the vascular sheath 124 positioned at the left lateral border of the pericardial sac 109. Vascular sheath 124 can be held stationary as spiral anchor catheter 100 is rotated to achieve placement through pericardium 109.

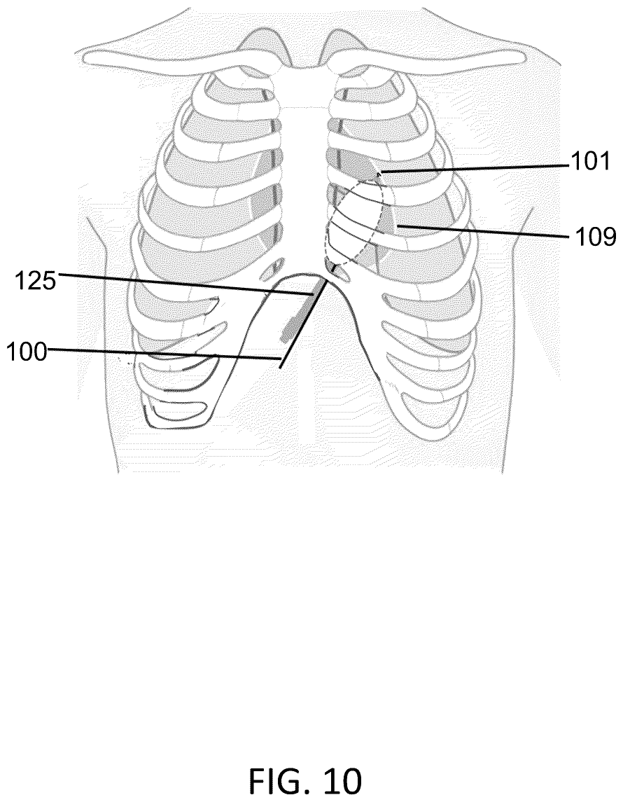

FIG. 10 shows advancement of the ventricular assist balloon cannula 125 along the shaft of spiral anchor catheter 100 after the spiral 101 has been fixated to the pericardial membrane 109. The ventricular assist balloon may be a component of a ventricular assist device as described herein.

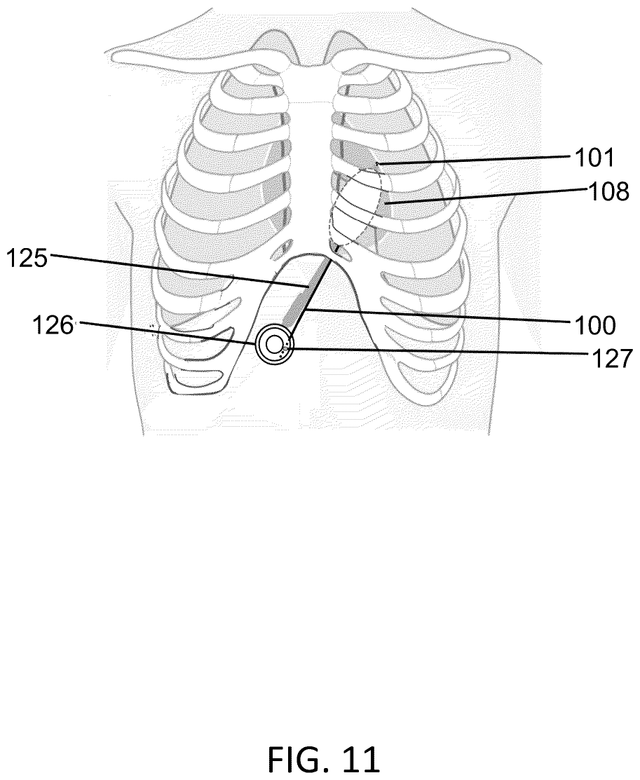

FIG. 11 shows that following advancement of ventricular assist balloon cannula 125 over spiral anchor catheter 100, a subcutaneous reservoir 126 may be attached to the proximal end of ventricular assist balloon cannula 125. The proximal end of spiral anchor catheter 100 may be inserted into a channel in the housing of subcutaneous reservoir 126, where it can be locked in position using a setscrew 127. The spiral 101 embedded in the pericardium 109 and the setscrew 127 fixation of the proximal spiral anchor catheter 100 to the reservoir 126 can prevent axial and transverse movement of the ventricular assist balloon cannula 125 with respect to the heart 108.

### Balloon

Described herein is a balloon cannula, sometimes referred to as a balloon catheter, that can be configured to cause or achieve cardiac compression (e.g., ventricular wall displacement) upon balloon inflation. The ability of the balloon cannula to achieve effective ventricular compression may be due to several factors, including balloon size, balloon shape, balloon material, anatomical balloon location, inflation medium, and other factors.

Balloon size can impact the percent of baseline cardiac output increase achieved by the cardiac assist device. Normal left ventricular stroke volume may be approximately 75 cc. The frequency of balloon inflation should be at least equal to a physiologic heart rate, approximately 70 bpm. A 75 cc volume balloon may achieve a modest cardiac output, measured via a digital flow transducer at the aortic outlet. In some cases, the balloon volume can be increased, either through additional inflation, a larger balloon, or both. The balloon volume can be increased to about 80 cc, about 85 cc, about 90 cc, about 95 cc, about 100 cc, about 105 cc, about 110 cc, about 115 cc, about 120 cc, about 125 cc, about 130 cc, about 135 cc, about 140 cc, or greater than about 140 cc. When the balloon volume is increased to about 120 cc, balloon inflation at 60 bpm can yield a cardiac output of about 100 ml/min, equivalent to 50% of the baseline cardiac output achieved via open heart massage at 60 bpm. In some embodiments, a balloon with an inflated volume of about 120 cc can provide a ventricular wall displacement of about 5.0 cm. In some embodiments, a balloon with an inflated volume of from about 100 cc to 140 cc can provide a ventricular wall displacement of about 3.0 cm to about 7 cm. In some embodiments, a balloon with an inflated volume of about 120 cc can provide a ventricular wall displacement of about 3 cm to about 4 cm, about 3 cm to about 5 cm, about 3 cm to about 6 cm, about 3 cm to about 7 cm, about 4 cm to about 5 cm, about 4 cm to about 6 cm, about 4 cm to about 7 cm, about 5 cm to about 6 cm, about 5 cm to about 7 cm, or about 6 cm to about 7 cm.

In some embodiments, a balloon with an inflated volume of about 120 cc can provide a ventricular wall displacement of less than about 3 cm, less than about 4 cm, less than about 5 cm, less than about 6 cm, or less than about 7 cm. In some embodiments, a balloon with an inflated volume of about 120 cc can provide a ventricular wall displacement of greater than about 3 cm, greater than about 4 cm, greater than about 5 cm, greater than about 6 cm, or greater than about 7 cm.

Balloon shape can impact the percent of baseline cardiac output increase achieved by the cardiac assist device. In some cases, the spherical balloon can achieve optimal cardiac output while maintaining its intrapericardial ventricular position without exhibiting atrial compression. The ventricular compression balloon may have a length sized to extend along the major portion of the ventricular cavity.

Balloon material can impact the percent of baseline cardiac output increase achieved by the cardiac assist device. In some embodiments, the balloon material comprises an inelastic polyethylene terephthalate (PET) material. In some embodiments, the balloon material comprises a partially elastic polyurethane. In some cases, the balloon material may comprise a single material (e.g., polyurethane). In some embodiments, the balloon material can comprise additional partially elastic materials, such as silicone rubber. The partially elastic polyurethane can have superior fatigue characteristics, and it can be expected that a balloon comprising the partially elastic polyurethane can last over one year for use as destination therapy. In some cases, lasting for a period of time can mean lasting for a period of time without breakage of the balloon. In some cases, the balloon can last more than about 1 year, more than about 2 years, more than about 3 years, more than about 4 years, more than about 5 years, more than about 6 years, more than about 7 years, more than about 8 years, more than about 9 years, or more than about 10 years. In some cases, the balloon can last from about 1 year to about 2 years, about 1 year to about 3 years, about 1 year to about 4 years, about 1 year to about 5 years, about 1 year to about 10 years, about 1 year to about 15 years, about 2 years to about 3 years, about 2 years to about 4 years, about 2 years to about 5 years, about 2 years to about 10 years, about 2 years to about 15 years, about 3 years to about 4 years, about 3 years to about 5 years, about 3 years to about 10 years, about 3 years to about 15 years, about 4 years to about 5 years, about 4 years to about 10 years, about 4 years to about 15 years, about 5 years to about 10 years, about 5 years to about 15 years, or from about 10 years to about 15 years. One year of balloon life implanted in a patient can result in about 40 million inflation cycles, where an inflation cycle can include full inflation and deflation. Polyurethane balloons may withstand more than about 60 million cycles.

In some cases, the balloon can comprise a material hardness of from about 75 Shore A durometers to about 95 Shore A durometers. In some cases, the balloon can comprise a material hardness of from about 75 Shore A durometers to about 80 Shore A durometers, about 75 Shore A durometers to about 85 Shore A durometers, about 75 Shore A durometers to about 90 Shore A durometers, about 75 Shore A durometers to about 95 Shore A durometers, about 80 Shore A durometers to about 85 Shore A durometers, about 80 Shore A durometers to about 90 Shore A durometers, about 80 Shore A durometers to about 95 Shore A durometers, about 85 Shore A durometers to about 90 Shore A durometers, about 85 Shore A durometers to about 95 Shore A durometers, or from about 90 Shore A durometers to about 95 Shore A durometers. In some cases, the balloon can comprise a material hardness of about 75 Shore A durometers, about 80 Shore A durometers, about 85 Shore A durometers, about 90 Shore A durometers, or about 95 Shore A durometers. In some cases, the balloon can comprise a material hardness of at least about 75 Shore A durometers, about 80 Shore A durometers, about 85 Shore A durometers, or about 90 Shore A durometers. In some cases, the balloon can comprise a material hardness of at most about 80 Shore A durometers, about 85 Shore A durometers, about 90 Shore A durometers, or about 95 Shore A durometers.

In some cases, the elastic modulus of the balloon material can be from about 4.5 MPa to about 7.5 MPa. In some cases, the elastic modulus of the balloon material can be from about 4.5 MPa to about 5 MPa, about 4.5 MPa to about 5.5 MPa, about 4.5 MPa to about 6 MPa, about 4.5 MPa to about 6.5 MPa, about 4.5 MPa to about 7 MPa, about 4.5 MPa to about 7.5 MPa, about 5 MPa to about 5.5 MPa, about 5 MPa to about 6 MPa, about 5 MPa to about 6.5 MPa, about 5 MPa to about 7 MPa, about 5 MPa to about 7.5 MPa, about 5.5 MPa to about 6 MPa, about 5.5 MPa to about 6.5 MPa, about 5.5 MPa to about 7 MPa, about 5.5 MPa to about 7.5 MPa, about 6 MPa to about 6.5 MPa, about 6 MPa to about 7 MPa, about 6 MPa to about 7.5 MPa, about 6.5 MPa to about 7 MPa, about 6.5 MPa to about 7.5 MPa, or from about 7 MPa to about 7.5 MPa. In some cases, the elastic modulus of the balloon material can be about 4.5 MPa, about 5 MPa, about 5.5 MPa, about 6 MPa, about 6.5 MPa, about 7 MPa, or about 7.5 MPa. In some cases, the elastic modulus of the balloon material can be at least about 4.5 MPa, about 5 MPa, about 5.5 MPa, about 6 MPa, about 6.5 MPa, or about 7 MPa. In some cases, the elastic modulus of the balloon material can be at most about 5 MPa, about 5.5 MPa, about 6 MPa, about 6.5 MPa, about 7 MPa, or about 7.5 MPa. In some cases, the elastic modulus of the balloon material can be from about 4.7 MPa in a wet environment at body temperature to about 7.4 MPa in a dry environment at room temperature.

A partially elastic balloon can also inflate with a cushioning effect on the myocardium, as opposed to a rigid balloon. This occurs because the partial elasticity allows for a small amount of “give” when the heart pushes back. The yielding can allow such balloons to be safer for more delicate hearts. The softness of a partially elastic balloon can be advantageous due to reduced impulse during balloon inflation and softer contact on the myocardium, which may decrease fibrosis and scarring of the myocardium with long term use of balloon cardiac augmentation. This may be important as a significant fraction of patients present with acute myocardial infarction.

Anatomical balloon location can impact the percent of baseline cardiac output increase achieved by the cardiac assist device. The balloon can be situated anterior to the left ventricle, in order to utilize the rigid bony enclosure formed by the sternum, ribcage and spine to achieve reliable ventricular compression in the anterior-posterior axis. In some embodiments, the anterior-posterior dimension may be preserved, which can reduce confounding effects of pericardial compliance and cardiomegaly.

Inflation medium can impact the percent of baseline cardiac output increase achieved by the cardiac assist device. The ventricular compression balloon may be inflated with fluid, either gas or liquid, usually being a gas in order to decrease the inflation/deflation times. A gas may be preferred, as it allows a fast response time for balloon inflation and balloon deflation. In some embodiments, air inflation may be used, which can facilitate the flow dynamics and provide the system with a frequency response that accommodates a heart rate up to about 120 bpm. Balloon inflation can be limited to the systolic cycle, with vacuum balloon deflation performed during diastole to ensure that ventricular refilling is not compromised. In some cases, air is allowed to naturally flow out during diastole by stopping inflation and allowing the pressure of the heart on the balloon to push the air out.

### Systems

Described herein is an implantable cardiac assist system. The system can comprise a cardiac assist catheter, a pneumatic effector, a reservoir, an exterior drive, a pump, and other components as described herein.

The pneumatic effector can comprise a ventricular compression balloon. The ventricular compression balloon may be inflated and deflated by means of an external battery-operated pump that resides outside of the patient's body. The pump may be bi-directional in flow, with an attached fluid reservoir. In the case of air inflation of the ventricular compression balloon, the pump inlet and outlet may vent directly to room air. Ventricular compression balloon inflation can be synchronized to the cardiac cycle by means of an electrical sensor that senses the patient's ECG (electrocardiographic) signal and initiates balloon inflation at the start of the QRS complex of the heart. Patient ECG sensing may be performed via a conductive needle from the external unit that punctures the patient's skin to perform balloon inflation. Alternatively, or in addition, ECG electrodes or other sensors may be disposed in the implantable port and/or catheter, and the needle used to provide connection to ECG circuitry in the external controller. A control unit in the external pump module can receive the ECG signal and trigger balloon inflation during systole. Active balloon deflation can be performed by the pump during diastole. Balloon inflation may also be performed using a hydraulic system that contains a pressurized fluid tank that provides balloon inflation during systole, and a pressure release valve that allows balloon deflation during diastole. The fluid tank may be re-pressurized periodically, or a battery-operated compressor may be part of the unit that resides outside the patient's body.

A minimum of two electrodes may be used to obtain an ECG signal. An ECG signal obtained from a needle inserted through body tissue may be superior to an ECG signal obtained from a surface electrode as the needle is less susceptible to motion artifacts and noise than a surface electrode. In some cases, one or more subcutaneous electrodes advanced over the chest wall from the subxiphoid incision. In some cases, one, two, or more electrodes may be located in the implantable reservoir, with a conductive mesh extending from each electrode to provide an expanded target to accommodate needle placement. The dimensions of the opening in the mesh can be an interference fit with the circumference of the needle; such that upon needle insertion, the mesh applies compressive force against the outer surface of the needle for ECG signal conduction. The needle may contain a series of bulbs or barbs to facilitate anchoring in the mesh component of the target electrode, preventing inadvertent needle detachment.

The body of the reservoir may be constructed of an implantable polymer material such as polycarbonate or polyvinyl chloride. The electrodes may be constructed of conductive metal such as stainless steel. If a single electrode is incorporated into one reservoir, the electrode may be a metallic ring attached to the outer diameter of the polymer reservoir. If two or more electrodes can be incorporated into a single reservoir, at least one electrode may be attached to the outer diameter of the reservoir, and at least one other electrode may be a small diameter concentric metal ring attached to the elastomeric face of the reservoir. A stainless-steel mesh may extend radially inward from the outer electrode towards the inner electrode, with a circumferential gap existing between the inner edge of the mesh and the outer edge of the inner electrode, to act as an insulator between the two electrodes. Alternatively, or in addition, the two electrodes may be formed by two diametrically opposed arcs on the outer diameter of the reservoir, with the steel mesh extending between the two ends of each arc, preserving a non-conductive area between the two conductive mesh regions. A combination of multiple electrodes on multiple reservoirs may be applied; for example, two electrodes may reside on one implanted reservoir, and a single electrode may reside on a second implanted reservoir to yield a three-lead EKG sensing array.

In some embodiments, the circuit board receiving the EKG signal and controlling activation of the external pump unit is positioned in the reservoir. The signal from the control board can be transmitted to the external pump unit via fiberoptic transmission, ensuring minimal signal noise due to patient motion. A fiberoptic connection consisting of a circumferential array around one of the inflation needles can mate with a corresponding circular array in the circuit board. A funnel guide can be incorporated into the inner concentric electrode of the reservoir so that needle insertion results in coaptation of the respective fiberoptic arrays on the needle and the control board in the reservoir. The two needles inserted through the mesh electrodes may carry current from the external unit to power the circuit board inside the reservoir.

In some cases, the present disclosure provides a cardiac assist system comprising a pneumatic effector configured to be implanted beneath a patient's pericardial sac and over a myocardial surface, typically overlying the patient's left ventricle. The system can further comprise an implantable port configured to receive a percutaneously introduced cannula, where the cannula supplies a driving gas to the pneumatic effector. The pneumatic effector may be a balloon located near a distal end of a catheter or other tubular body, where the implantable port can be connected to the catheter or other tube near a proximal end.

The cardiac assist system can further comprise an external drive unit which includes a pump assembly and control circuitry. The pump assembly can include at least one pump for delivering a gas, for example ambient air, to the cannula. The control circuitry can be configured to operate the pump to actuate the pneumatic effector in response to the patient's sensed heart rhythm. The connecting tube can have a pump end which is attachable to the pump of the pump assembly and a cannula end attached to the cannula.

In some embodiments, the pneumatic effector may comprise an inflatable bladder, such as a medical balloon, for example a relatively non-distensible medical balloon on a catheter, where the balloon is configured to sit over the left ventricle from the patient and beneath the inner surface of the pericardial sac. The balloon can have a volume, when fully inflated, in a range from about 50 ml to about 200 ml. The balloon can have a volume, when fully inflated, in a range from about 50 ml to about 80 ml, about 50 ml to about 110 ml, about 50 ml to about 140 ml, about 50 ml to about 170 ml, about 50 ml to about 200 ml, about 80 ml to about 110 ml, about 80 ml to about 140 ml, about 80 ml to about 170 ml, about 80 ml to about 200 ml, about 110 ml to about 140 ml, about 110 ml to about 170 ml, about 110 ml to about 200 ml, about 140 ml to about 170 ml, about 140 ml to about 200 ml, or about 170 ml to about 200 ml. The balloon can have a volume, when fully inflated, less than about 50 ml, less than about 80 ml, less than about 110 ml, less than about 140 ml, less than about 170 ml, or less than about 200 ml. The balloon can have a volume, when fully inflated, greater than about 50 ml, greater than about 80 ml, greater than about 110 ml, greater than about 140 ml, greater than about 170 ml, or greater than about 200 ml.

In some cases, the volume of the balloon when fully inflated can be from about 76 ml to about 125 ml. In some cases, the volume of the balloon when fully inflated can be from about In some cases, the volume of the balloon when fully inflated can be from about 76 ml to about 83 ml, about 76 ml to about 90 ml, about 76 ml to about 97 ml, about 76 ml to about 104 ml, about 76 ml to about 111 ml, about 76 ml to about 118 ml, about 76 ml to about 125 ml, about 83 ml to about 90 ml, about 83 ml to about 97 ml, about 83 ml to about 104 ml, about 83 ml to about 111 ml, about 83 ml to about 118 ml, about 83 ml to about 125 ml, about 90 ml to about 97 ml, about 90 ml to about 104 ml, about 90 ml to about 111 ml, about 90 ml to about 118 ml, about 90 ml to about 125 ml, about 97 ml to about 104 ml, about 97 ml to about 111 ml, about 97 ml to about 118 ml, about 97 ml to about 125 ml, about 104 ml to about 111 ml, about 104 ml to about 118 ml, about 104 ml to about 125 ml, about 111 ml to about 118 ml, about 111 ml to about 125 ml, about 118 ml to about 125 ml. In some cases, the volume of the balloon when fully inflated can be less than about 76 ml, less than about 83 ml, less than about 90 ml, less than about 97 ml, less than about 104 ml, less than about 111 ml, less than about 118 ml, or less than about 125 ml. In some cases, the volume of the balloon when fully inflated can be greater than about 76 ml, greater than about 83 ml, greater than about 90 ml, greater than about 97 ml, greater than about 104 ml, greater than about 111 ml, greater than about 118 ml, or greater than about 125 ml.

In some cases, the pneumatic effector may comprise some other pneumatically actuable mechanical device, such as a piston and cylinder arrangement.

In some cases, the implantable port may comprise a needle-penetrable septum, where the cannula comprises a needle or other sharpened tube or hollow probe configured to percutaneously penetrate the patient's tissue overlying the septum, for example the abdominal wall. The needle or other sharpened tube or hollow probe can further penetrate the septum to fluidly connect the pump assembly and the external drive unit with the interior of the port so that the air or other gas may be delivered under pressure to inflate or otherwise actuate the pneumatic effector.

In some cases, the septum can have an area which is sufficiently large to provide multiple sites for needle penetration. This is a particular advantage when a percutaneous insertion site becomes infected. By having multiple needle penetration sites, the needle may be removed from an infected area, the patient treated, and a new or sterilized needle introduced to the implantable port through an alternate site on the septum. In this way, one can avoid explanting neither or otherwise significantly disturbing the implantable port nor the pneumatic effector to treat the infection.

In some cases, the implantable port may comprise a mechanical valve for receiving the needle or other cannula. Such mechanical ports can assist with hemodialysis access and other purposes.

In some embodiments, the system can further comprise at least one electrocardiogram (ECG) electrode located and configured to detect the patient's cardiac rhythm. The ECG electrode may be located externally as with conventional ECG detection systems, and may comprise one, two, three, four, or more external electrodes. One or more ECG electrodes may be incorporated into the cardiac assist system. For example, the electrodes may be provided by or incorporated into the needle or other cannula used to access the implantable port. Alternatively, or additionally, one or more ECG electrodes may be located on or coupled to the implantable port itself. For example, one, two, three, four, or more ECG electrodes may be attached to an exterior surface of the port housing, the port septum or other membrane, or elsewhere on the port. Further alternatively or additionally, one or more ECG electrodes may be located on the connecting tube and/or on the pneumatic effector itself. In some embodiments, the at least one ECG electrode may be located in subcutaneous tissue.

In systems having ECG electrodes, the external drive unit can further comprise ECG circuitry in order to detect cardiac rhythm and synchronize actuation of the pump assembly with the cardiac rhythm. When the ECG electrode(s) can be present on any portion of the implantable port or connecting tube, electrical conductors may be provided in or through the needle or other cannula in order to deliver the electrical signals to the ECG circuitry. When the needle or other cannula itself comprises the ECG electrode, the needle or other cannula may be connected to the ECG circuitry through a conductor in the tube or other structure which connects the cannula to the external electrode. External ECG electrodes may be connected to the external controller by conventional ECG leads, for example external leads.

The control circuitry in the external drive unit can be configured to actuate the pneumatic effector in synchrony with the patient's cardiac rhythm as measured by the ECG. For example, the control circuitry may be configured to actuate the pneumatic effector at each occurrence of an R wave peak. The control circuitry may be further configured to detect abnormal cardiac rhythms. For example, the control circuitry may compare the time between successive individual R peaks and determine the occurrence of an abnormal cardiac rhythm based upon time increases or decreases by more than a predetermined threshold percentage, such as about 300%. In some cases, the predetermined threshold percentage can be about 250%. In some cases, the predetermined threshold percentage can be from about 225% to about 245%, about 225% to about 265%, about 225% to about 285%, about 225% to about 305%, about 225% to about 325%, about 245% to about 265%, about 245% to about 285%, about 245% to about 305%, about 245% to about 325%, about 265% to about 285%, about 265% to about 305%, about 265% to about 325%, about 285% to about 305%, about 285% to about 325%, or about 305% to about 325%. In some cases, the predetermined threshold percentage can be less than about 220%, less than about 230%, less than about 240%, less than about 250%, less than about 260%, less than about 270%, less than about 280%, less than about 290%, less than about 300%, less than about 310%, or less than about 320%. In some cases, the predetermined threshold percentage can be greater than about 220%, greater than about 230%, greater than about 240%, greater than about 250%, greater than about 260%, greater than about 270%, greater than about 280%, greater than about 290%, greater than about 300%, greater than about 310%, or greater than about 320%.

Alternatively, or additionally, the control circuitry may compare the time between successive cumulative R peaks and determine whether an abnormal cardiac rhythm exists based upon an aggregate variability exceeding a predetermined threshold percentage, such as about 300%. In some cases, the predetermined threshold percentage can be about 250%. In some cases, the predetermined threshold percentage can be from about 225% to about 245%, about 225% to about 265%, about 225% to about 285%, about 225% to about 305%, about 225% to about 325%, about 245% to about 265%, about 245% to about 285%, about 245% to about 305%, about 245% to about 325%, about 265% to about 285%, about 265% to about 305%, about 265% to about 325%, about 285% to about 305%, about 285% to about 325%, or about 305% to about 325%. In some cases, the predetermined threshold percentage can be less than about 220%, less than about 230%, less than about 240%, less than about 250%, less than about 260%, less than about 270%, less than about 280%, less than about 290%, less than about 300%, less than about 310%, or less than about 320%. In some cases, the predetermined threshold percentage can be greater than about 220%, greater than about 230%, greater than about 240%, greater than about 250%, greater than about 260%, greater than about 270%, greater than about 280%, greater than about 290%, greater than about 300%, greater than about 310%, or greater than about 320%.

When the control circuitry detects an abnormal cardiac rhythm, it may take one or more of several actions. For example, the control circuitry may simply stop actuation of the pneumatic effector until the cardiac rhythm returns to a normal pattern. In some cases, the control circuitry may actuate the pneumatic effector at a predetermined fixed rate, such as a fixed rate in the range from about 50 beats per minute (bpm) to about 80 bpm and continue such actuation until a normal cardiac rhythm is reestablished. In some cases, the control circuitry may be configured to actuate the pneumatic effector at a “proportionally” modified rate relative to the patient cardiac rhythm when the abnormal cardiac rhythm is detected. That is, while the actuation of the pneumatic effector may be at 1:1 ratio when the cardiac rhythm is normal, when an abnormal cardiac rhythm is detected, the rate may be changed to another ratio. For example, if the patient experiences an abnormally high heart rate (tachycardia), then the rate at which the pneumatic effector is triggered may be decreased. The pneumatic effector may be triggered once for every two heartbeats (1:2), once for every three heartbeats (1:3), more frequently, less frequently, or the like.

In some embodiments, the pump assembly of the cardiac assist system may be configured to alternately deliver the driving gas to the pneumatic effector and to withdraw the driving gas from the pneumatic effector. Each cycle of delivery and withdrawal may correspond to a single trigger event from the control circuitry of the external controller. The cycle of pump delivery and pump withdrawal can correspond to the patient's heartrate as detected from the ECG, with a single cycle typically lasting about one second for a patient with a heartrate of 60 bpm. Such rapid inflation and deflation can use a fast-acting system, and the available gas delivery volumes, fluid transmission lumen dimensions, and the like, can conform to the inflation and deflation times. To achieve the rapid delivery and extraction times, the fluid being delivered may be a gas such as, for example, ambient air.

The pump assembly may take any one of a variety of configurations for delivering and withdrawing the fluid to the pneumatic effector. For example, the pump assembly may include a single pump which operates in a single flow direction, further comprising valving necessary to divert the gas from an inflation direction to a deflation direction. In some embodiments, the pump assembly can comprise one pump connected to valving, wherein the pump is configured to flow driving gas in one direction and the valving is configured to alternately deliver the driving gas to the pneumatic effector and withdraw the driving gas from the pneumatic effector.

Alternatively, or in addition, the pump assembly may comprise two or more pumps, at least one operating in a fluid delivery direction and at least one operating in a fluid extraction direction, for example one to deliver driving gas and one to remove driving gas from the pneumatic effector. The pump assembly may connect to either a single cannula which provides for both fluid delivery and fluid extraction from the implantable pump. Alternatively, or in addition, the pump assembly may be connected to two or more cannulas, one or more for delivering the inflation fluid and one or more for extracting the inflation fluid.

Disclosed herein is an implantable cardiac assist catheter for use with an external drive unit, wherein the implantable cardiac assist catheter can comprise a catheter body having a proximal and a distal end. A pneumatic effector can be attached to the distal end of the catheter body and be configured to be implanted beneath a patient's pericardial sac and over a myocardial surface overlying the patient's left ventricle. An implantable port can be attached at a proximal end of the catheter body and be configured to receive a percutaneously introduced cannula. The port can be connected to supply a driving gas received from the cannula through a gas lumen in the catheter body to the pneumatic effector. By controlling the supply of driving gas to the implantable port, the pneumatic effector can be driven at a desired rate as controlled by the external drive unit.

In some embodiments, the implantable cardiac assist catheter may comprise a distal tip having a guidewire lumen with an entry port and an exit port, where both ports can be located distal to the pneumatic effector. Such “monorail” construction can be advantageous because it can allow the remainder of the catheter body to have only a single fluid delivery lumen, thus reducing the needed diameter of the catheter. Thus, the gas lumen and the catheter body may be the only lumen present between the proximal end of the catheter body and the pneumatic effector.

In some cases, the implantable cardiac assist catheter may comprise a distal section or distal tip having a guidewire lumen with an entry port and an exit port. One port can be located proximal to the pneumatic effector, and the other port can be located distal to the pneumatic effector. In some cases, a guidewire lumen that contains a length near the length of the balloon, with an entry port proximate and distal to the balloon, and an exit port proximate and proximal to the balloon, can confer multiple advantages. The guidewire lumen may accommodate the shaft of the spiral anchor wire, and can serve the purpose of maintaining the position of the balloon over the heart while allowing a limited amount of axial motion of the balloon catheter along the anchor wire to avoid traction on the spiral anchor embedded in the lateral aspect of the pericardium during each cycle of balloon inflation. Traction on the spiral anchor during balloon inflation may cause spiral anchor dislodgement and loss of balloon positioning. A guidewire lumen that extends the length of the balloon instead of the full length of the catheter can allow the catheter body to assume a smaller outer diameter profile. It can also reduce the friction encountered during advancement of the balloon catheter along the anchor wire, facilitating the process of balloon catheter insertion.

In some embodiments, the gas lumen is the only lumen in the catheter body between the proximal end and the pneumatic effector. In some embodiments, the implantable cardiac assist catheter further includes a guidewire lumen in its distal section to accommodates an anchor wire attached to the pericardium. An anchor wire can be anchored to the lateral aspect of the pericardium, and the guidewire lumen of the implantable assist catheter can be advanced over the anchor wire into position anterior to the heart, stabilizing the catheter position for subsequent use.

Provided herein is an external drive unit for use with an implantable cardiac assist catheter, such as the cardiac assist catheter just described. The external driving unit can comprise a pump assembly and control circuitry. The control circuitry can be configured to operate the pump assembly to actuate a pneumatic effector on the implantable cardiac assist catheter in response to the patient's sensed heart rhythm.

In some embodiments, the external drive unit may further comprise a connecting tube having a pump and connected to the pump assembly. The external drive unit may further comprise a percutaneous port-connecting end configured to be removably attached to an implantable port fluidly connected to the pneumatic effector on the implantable cardiac assist catheter. In some embodiments, the external drive unit may include ECG circuitry. The ECG circuitry may be configured to receive signals from at least one ECG electrode, located in any of the locations described previously. The at least one ECG electrode may be implanted to detect the patient's cardiac rhythm. The connecting tube can comprise at least one conductor electrically coupled to the cannula and be configured to connect ECG electrodes to the ECG circuitry in the external drive unit.

In some embodiments, the control circuitry of the external drive unit may be configured to actuate the pneumatic effector in synchrony with the patient's cardiac rhythm as measured by an ECG electrode. Actuation may be accomplished by detecting the occurrence of R wave peaks, as described generally above. The controls circuitry may be configured to detect abnormal cardiac rhythms. The controls circuitry may be configured to stop or modify the actuation of the pneumatic effector in any of the ways described previously. The pump assembly may also have any of the configurations described previously with respect to the cardiac assist systems of the present disclosure.

Referring back to FIG. 1, a perspective view of a cardiac assist system 10 in accordance with example embodiments described herein is shown. Cardiac assist system 10 may comprise a cardiac assist catheter 12 and an external drive unit 14. The cardiac assist catheter 12 can include a catheter body 18 having a pneumatic effector 20 located at its distal end. The pneumatic effector can be a balloon. An implantable port 24 is connected at a proximal end of the catheter body 18, and the body can have a distal tip 26 with a short “monorail” guidewire lumen defined between a guidewire lumen entry port 28 and a guidewire lumen exit port 30.

The length of the guidewire lumen can be from about 0.5 cm to about 3 cm. The length of the guidewire lumen can be from about 0.5 cm to about 1 cm, about 0.5 cm to about 1.5 cm, about 0.5 cm to about 2 cm, about 0.5 cm to about 2.5 cm, about 0.5 cm to about 3 cm, about 1 cm to about 1.5 cm, about 1 cm to about 2 cm, about 1 cm to about 2.5 cm, about 1 cm to about 3 cm, about 1.5 cm to about 2 cm, about 1.5 cm to about 2.5 cm, about 1.5 cm to about 3 cm, about 2 cm to about 2.5 cm, about 2 cm to about 3 cm, or about 2.5 cm to about 3 cm. The length of the guidewire lumen can be less than about 0.5 cm, less than about 1 cm, less than about 1.5 cm, less than about 2 cm, less than about 2.5 cm, or less than about 3 cm. The length of the guidewire lumen can be greater than about 0.5 cm, greater than about 1 cm, greater than about 1.5 cm, greater than about 2 cm, greater than about 2.5 cm, or greater than about 3 cm.

The length of the guidewire lumen can be from about 1 cm to about 2 cm. The length of the guidewire lumen can be from about 1 cm to about 1.2 cm, about 1 cm to about 1.4 cm, about 1 cm to about 1.6 cm, about 1 cm to about 1.8 cm, about 1 cm to about 2 cm, about 1.2 cm to about 1.4 cm, about 1.2 cm to about 1.6 cm, about 1.2 cm to about 1.8 cm, about 1.2 cm to about 2 cm, about 1.4 cm to about 1.6 cm, about 1.4 cm to about 1.8 cm, about 1.4 cm to about 2 cm, about 1.6 cm to about 1.8 cm, about 1.6 cm to about 2 cm, or about 1.8 cm to about 2 cm. The length of the guidewire lumen can be less than about 1 cm, less than about 1.2 cm, less than about 1.4 cm, less than about 1.6 cm, less than about 1.8 cm, or less than about 2 cm. The length of the guidewire lumen can be greater than about 1 cm, greater than about 1.2 cm, greater than about 1.4 cm, greater than about 1.6 cm, greater than about 1.8 cm, or greater than about 2 cm.

The port 24 comprises a port body 38 or other enclosure having an opening on its upper surface. The opening can be covered by a needle-penetrable septum 40.

The external drive unit 14 can comprise a needle 44 or other cannula attached to a distal end of a connecting tube 46. The connecting tube can be attached to a pump 52 within a console 48, and the console further includes control circuitry 54 for controlling the pump and other operations of the cardiac assist system 10. The external drive unit 14 may further comprise an ECG pad 56 connected to the control circuitry 54 by a connecting cable 58. The pump, control circuitry, and all other active components can be battery operated or manually operated, and the external drive unit 14 can include a replaceable and/or rechargeable battery.

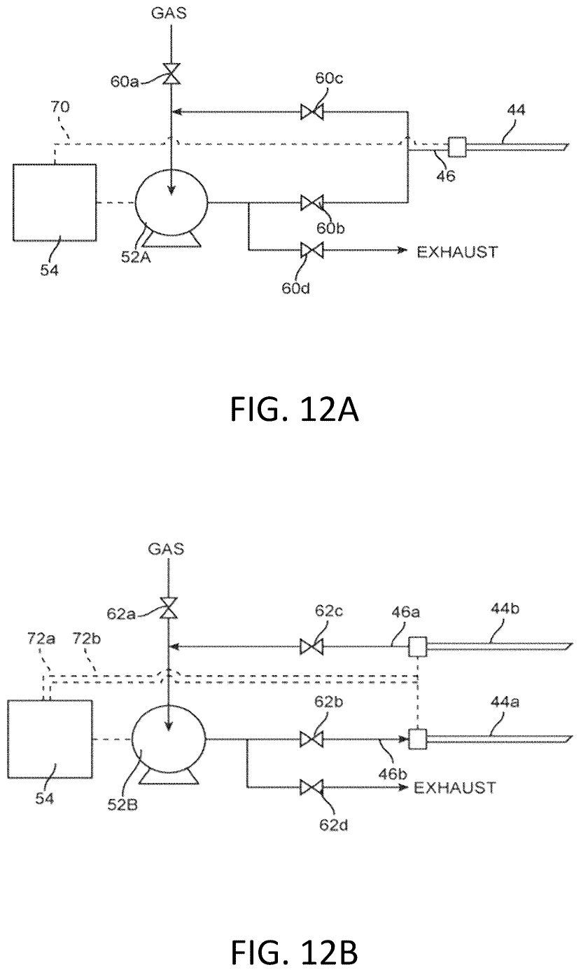

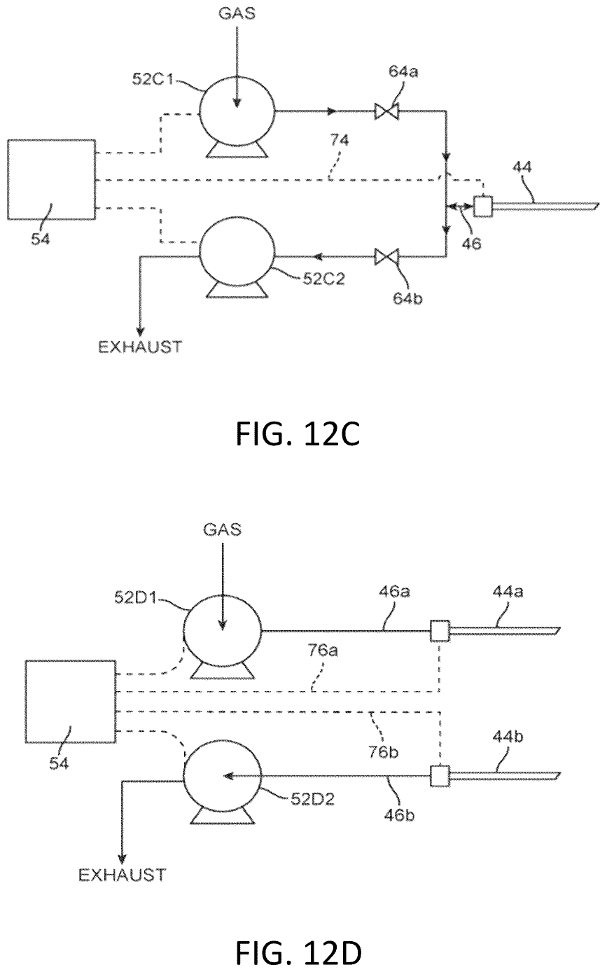

Referring now to FIGS. 12A-12D, which illustrate schematic views of configurations of a pump assembly suitable for use with cardiac assist systems in accordance with example embodiments described herein, the pump assembly 52 may comprise a variety of configurations. For example, as shown in FIG. 12A, a pump unit 52A, for example a diaphragm type of pump, can be connected to cannula 44 by a series of valves 60a-60d via connecting tube 46. The pump 50A can be configured to often run in a single direction, e.g., the inlet may receive gas and the outlet may deliver gas. To deliver gas to the cannula 44, valve 60a can be open, thereby allowing ambient air to flow to the pump 50A. The ambient air can be delivered out of the outlet of the pump to valve 60b which is open. When delivering gas to the cannula, valves 60c and 60d can be closed. Gas continues to be delivered until the pneumatic effector 20 is inflated, at which time the valves 60a-60d can be reversed. That is, gas inlet valve 60a may be closed and return bypass valve 60c may be opened, allowing gas to be extracted through the cannula 44 and delivered by the pump 52A through an open exhaust valve 60d. By reversing the open/closed status of valves 60a-60d, the pump can be caused to first deliver gas through the cannula and then extract gas through the cannula without reversing operation of the pump 52A.

As also shown in FIG. 12A, an ECG lead line 70 may be provided between the cannula 44 and the control circuitry 54 of the external drive unit.

FIG. 12B likewise comprises cannulas 44a and 44b, connecting tubes 46a and 46b, single pump 52B, control circuitry 54, valves 62a-62d, and ECG leads 72a and 72b. A single pump 52B may be used with a pair of cannulas 44a and 44b. Valves 62a-62d can be used to reverse gas flow between the cannulas. In particular, by opening valve 62a and valve 62d, ambient air may be delivered to gas delivery cannula 44a. By then closing valves 62a and 62b, and opening valves 62c and 62d, gas may be extracted through cannula 44b and out the exhaust valve 62d. Inflow and outflow of gas may be cycled by reversing the status of the valves in a manner similar to the valves of FIG. 12A.

In FIG. 12B, each cannula 44a and 44b can have a separate ECG lead 72a and 72b connected to the control circuitry 54 of the external drive unit.

Referring now to FIG. 12C, a pump assembly 52 can comprise two pumps 52c1 and 52c2. Control circuitry 54 can actuate the first pump 52c1 to deliver ambient air through valves 64ₐ to connecting tube 46 and then through cannula 44, while return valve 64b remains closed. After the pneumatic effector is fully inflated or otherwise actuated, valve 64a can be closed and the pump may be stopped. Return valve 64b can then be opened and pump 52c2 actuated to exhaust gas through the cannula 44 and out through the exhaust line, as illustrated. Operation of the two pumps and valves may be periodically reversed in order to cycle delivery and exhaust gas to and from the implanted port at a desired rate.

As also shown in FIG. 12C, an ECG lead line 74 may be provided between the cannula 44 and the control circuitry 54 of the external drive unit 14.

In some cases, with reference to FIG. 12D, a pump assembly can comprise two pumps 52d1 and 52d2. Each pump, in turn, may be connected to a single cannula 44a and 44b, respectively through connecting tubes 46a and 46b, respectively. While valving may be provided, the system can function without valves to cycle the system between gas delivery through pump 52d1 and cannula 44a and gas extraction through cannula 44b and pump 52d2 this may be achieved by simply starting and stopping the pumps at alternate times during the cycle.

As also shown in FIG. 12D, each cannula 44a and 44b can be connected to the control circuitry 54 by an ECG lead 76a and 76b, respectively.

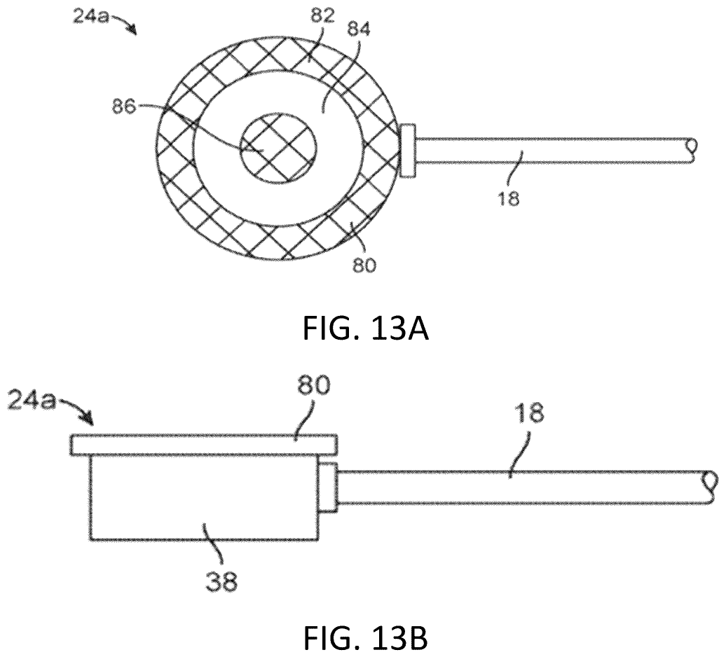

Referring now to FIGS. 13A and 13B, a first implantable port embodiment 24a is illustrated. FIG. 13A shows a top view of the port where the top surface can be oriented “anteriorly” toward the skin of the patient after it is implanted. FIG. 13B shows a side view of the port. A ring electrode 80 can be attached to the outer circumference of the implantable port 24a, located on port body 38, and catheter body 18. The ring electrode 80 may act as an ECG electrode. A circumferential band of metallic mesh 82 may be positioned over the outer perimeter of the upper surface of the port, typically over a needle-penetrable septum 84. A second, smaller inner ring electrode 86 may also be provided on an upper surface of the septum. The outer mesh electrode 82 can be electrically insulated from the inner electrode 86 by the nonconductive septum material. Thus, first and second metal cannulas may be introduced through the inner and outer electrodes to provide for separate connections for a two-lead EKG signal detection. The ECG electrodes, themselves, may be located on the body of the port 44a or elsewhere in the implantable system. Internal conductive wiring can be provided between the ECG electrode(s) and the mesh electrodes 82 and 86.

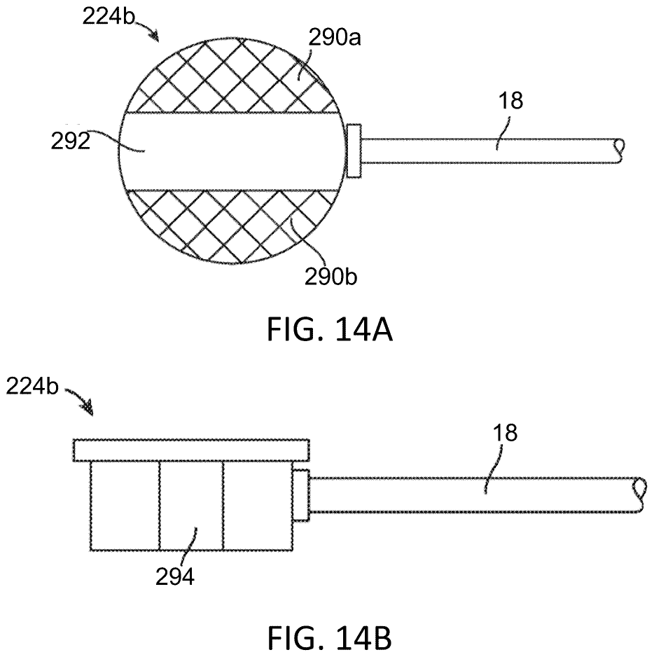

Referring now to FIGS. 14A and 14B, which illustrate top down and side views of a second implantable port embodiment, an alternative implantable port 224b is illustrated having first and second arcuate mesh electrodes 290a and 290b formed on an upper surface of a needle-penetrable septum 292 in FIG. 14A. As with FIGS. 13A-13B, the port can be disposed on a tip of the catheter body 18. As shown in FIG. 14B, a first electrode 294a may be positioned on an outer perimeter of the port body with an internal connection to one of the two arcuate mesh electrodes. A second electrode is located on the opposite face of the port body and may be connected to the other of the arcuate mesh electrodes in order to provide for separate cannula connections to each of the ECG electrodes.

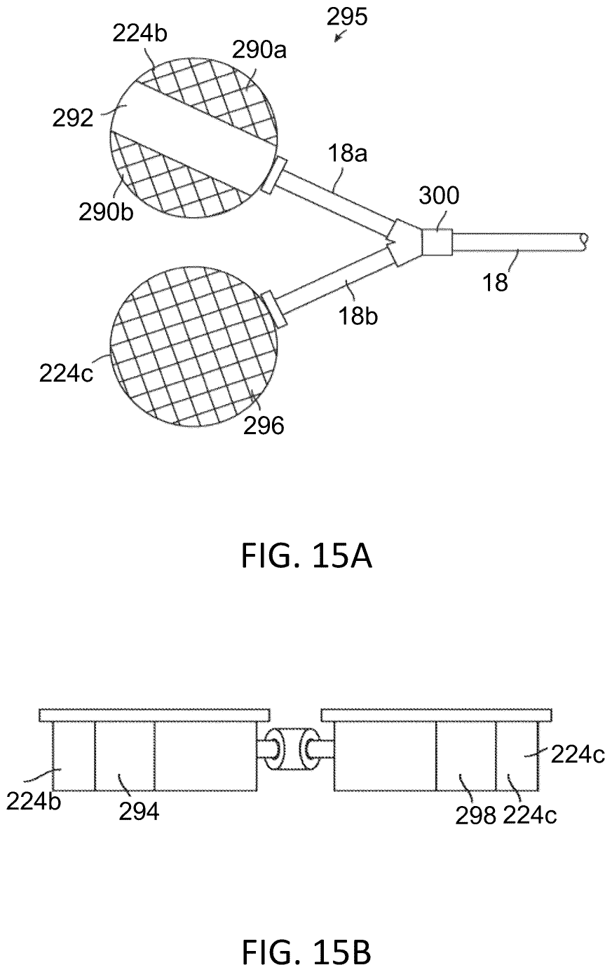

FIGS. 15A and 15B illustrate top down (FIG. 15A) and side views (FIG. 15B) of a third implantable port configuration 295. The port assembly 295 includes a first port 224b which may be similar to that described previously with respect to FIGS. 14A and 14B and comprise first and second arcuate mesh electrodes 290a and 290b formed on an upper surface of a needle-penetrable septum 292. A second port 224c may have a single mesh electrode covering the entire surface of an underlying needle-penetrable septum. The mesh electrode 296 may be connected to any ECG electrode, typically being connected to an electrically conductive portion of the port body. The ports 224b and 224c can be connected together through catheter portions 18a and 18b through a y-connector 300 to a common catheter body 18 which may be connected to the pneumatic effector as described previously with regard to FIG. 1.

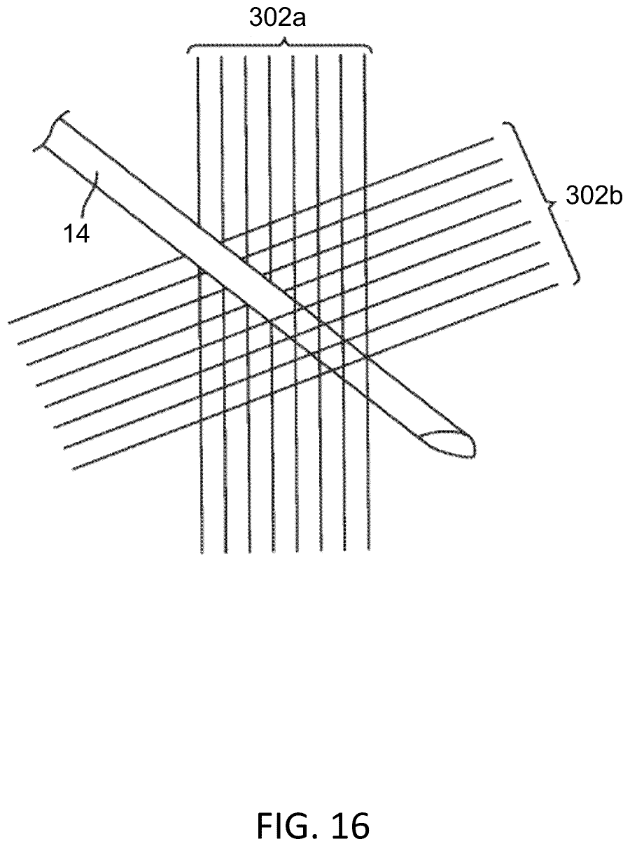

FIG. 16 illustrates how the cannula 14 forms an electrical connection with the wires of a mesh electrode. In particular, the mesh electrodes comprise orthogonally arranged conductive wires 302a and 302b. Small square-shaped cells can be formed where the wires 302a and 302b cross over. The dimensions of the cells can be chosen so that they can be smaller than the diameter of the cannula so that, when inserted through the wire mesh, the wall of the cannula 14 will necessarily contact all four wire which define a single cell. In this way, the frequency of good electrical contact can be increased.

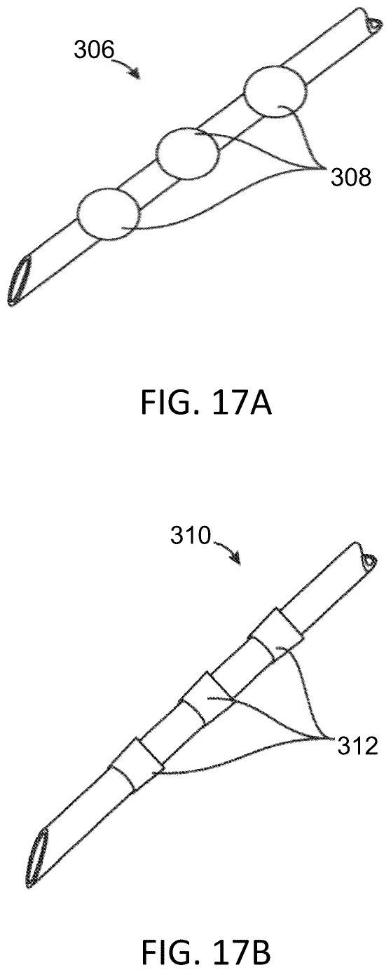

Referring now to FIG. 17A, the cannulas may be modified to enhance retention when they can be inserted through the septum of the implantable ports, particularly through the wire mesh electrode structures on such septum. In particular, as shown in FIG. 17A, a needle structure 106 may have a series of bulbs 308 which help the needle resist accidental extraction from the mesh. Similarly, as shown in FIG. 17B, needle 310 may have a series of barbs 312 which resist needle extraction.

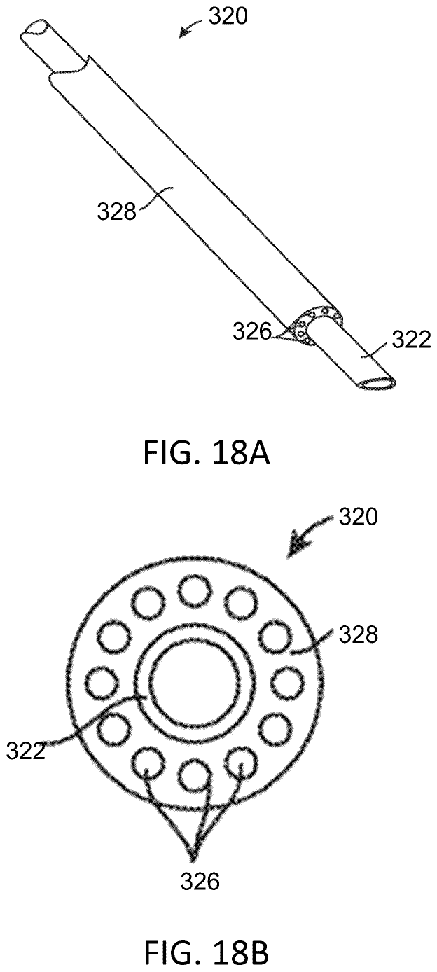

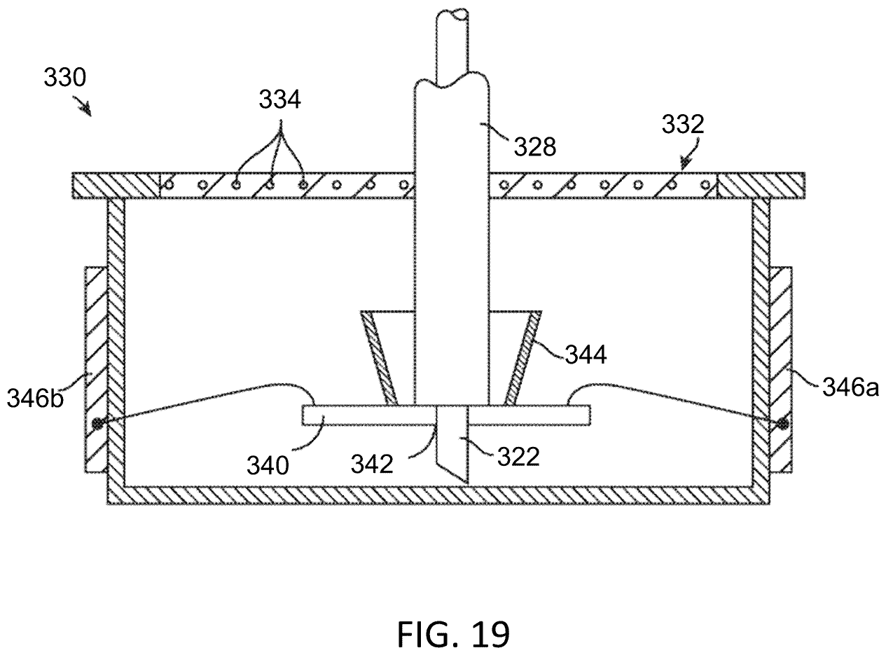

Referring now to FIGS. 18A, 18B, and 19, an optical system for delivering ECG information from an implantable port to the external drive unit can be described. As shown in FIG. 18A, a needle assembly 320 comprises a needle 322 having a plurality of optical fibers 326 embedded axially in a sheath 328. As shown in FIG. 18B, the optical fibers 326 can be exposed in a distal surface of the sheath 328 surrounding needle 322 in needle assembly 320. As shown in FIG. 19, an implantable port 330 may be modified to receive the needle assembly in a manner which can transmit optical information to the optical fibers. The needle assembly may be inserted through septum 332 of the port. An outer surface of the sheath 328 engages wires 334 of a grid embedded in the septum 332. Wires 334 can form the grid in the elastomer face of the subcutaneous reservoir. A distal surface of the sheath 328 can engage an upper surface of a circuit board 340. The circuit board 340 can comprise a plurality of optical emitters configured to deliver light to the optical fibers of the needle assembly. The needle 322 may pass through a hole or aperture 342 in the circuit board to permit the face of the sheath to engage the circuit board. A funnel 344 can be provided to assist in proper alignment of the needle with the aperture. ECG electrodes 346a and 146b on an exterior of the port 330 can be connected to the circuit board 140. Circuitry on the circuit board can extract ECG information from the electrodes and may convert that information to optical energy which is delivered by the optical emitters to the optical fibers 326. The light may be transmitted by the optical fibers to the external drive unit where it is converted back into electronic information suitable for controlling the system as described previously.

Referring now to FIG. 20, the cardiac assist system 10 of FIG. 1 may be implanted in a patient as illustrated. In particular, the balloon or other pneumatic effector 20 is introduced into the pericardial sac between an inner surface of the pericardium P and an outer surface of the myocardium M. The pneumatic effector 20 can be located generally over the left ventricle so that inflation or other actuation of the effector compresses the left ventricle, as shown in broken line in FIG. 20. Port 24 can be connected to the balloon by catheter body 18 and accessed percutaneously by cannula 14 through the abdominal wall AW. The external drive unit 48 can deliver actuating gas through connecting tube 46 and cannula 14 to the port or subcutaneous reservoir 24 to actuate the pneumatic effector, for example by inflating and deflating a balloon. In some cases, for examples when the pneumatic effector is not a balloon, actuating the pneumatic effector may comprise pumping it or otherwise increasing its size. An ECG signal can be captured by the ECG pad 56 which is connected to the external drive unit by cable 58. Other ECG signals may be captured by electrodes on the implantable port or subcutaneous reservoir 24 or elsewhere in the system. Pneumatic effector 20 can be stabilized by means of an anchor wire 59 anchored to the pericardium P and residing in a channel of catheter body 18 extending the length of pneumatic effector 20. Distal tip 26 can be located distal to the pneumatic effector 20.

Spiral anchor wire 59 attached to the pericardium P can enter the balloon catheter 18 via a port distal to balloon 20, and can exit the balloon catheter via a port proximal to balloon 20, preventing the tip of the catheter 26 from migrating out of position over the myocardium M. The proximal end of the anchor wire 59 attaches to the subcutaneous reservoir 24.

Anchor wire 59 can comprise an elongated wire with a spiral at the distal tip, wherein the distal tip comprises the anchor and the wire component can be used to bring the catheter near the spiral anchor.

### Methods

Disclosed herein are methods for implanting and using cardiac assist catheters as described herein.

### Methods for Implanting a Cardiac Assist Catheter

Described herein can be percutaneous and surgical methods for implanting a cardiac assist device as disclosed herein. Access to the pericardial sac can be performed via at least two alternative methods.

In the first method, a percutaneous, non-surgical pericardial entry is conducted using a subxiphoid needle insertion using a long, small bore hypodermic needle. In some cases, this is a 21-gauge needle. Upon successful needle entry through the pericardial membrane, a guidewire can be inserted into the pericardial sac and the hypodermic needle is removed, leaving the guidewire in place. An articulating vascular sheath with an inner tapered dilator can be advanced over the guidewire, expanding the pericardial puncture site and allowing the spiral anchor wire to be inserted and attached to the lateral aspect of the pericardium. The articulating sheath can contain a distal end that may be formed into a 90-degree bend, controlled by the physician. It may allow the anchor wire to contact the pericardial membrane in an orthogonal orientation to facilitate rotational entry of the sharp distal spiral. The spiral anchor can remain in position while the articulating sheath is removed. In some cases, a larger vascular sheath containing the balloon catheter is advanced into the pericardial sac over the anchor wire. A dedicated guidewire lumen that extends the full length of the balloon can accommodate the anchor wire. Following balloon placement, the delivery sheath may be removed, leaving the cardiac assist balloon in position.

In a second method, pericardial access may be performed surgically, via a subxiphoid incision instead of hypodermic needle insertion. This can be referred to as a “pericardial window,” and it may involve surgical dissection of a tract through the abdominal wall and the diaphragm to visualize, grasp and enter the pericardium. The xiphoid process, the inferior most cartilaginous portion of the sternum, may or may not be resected in a pericardial window procedure. Surgical retractors can retract the soft tissue along the sides of the tract to enhance visualization of the anatomy leading to the pericardium. Once the pericardium is grasped using long surgical clamps or forceps, it can be incised in a controlled fashion, and the articulating vascular sheath inserted for anchor wire placement. The remainder of the method can be similar to the first method. The remainder of the method can have differences from the first method.

These two methods are further discussed below and in the examples.

Minimally invasive access to the pericardium and heart for placement of the ventricular compression device can be performed in the subxiphoid region of the patient. The xiphoid process is the inferior most portion of the sternum, and myocardial access via subxiphoid entry can avoid the need for large sternotomy or thoracotomy incisions. Subxiphoid pericardial entry may be either conducted percutaneously, using needle pericardial entry followed by insertion of larger diameter vascular sheaths with tapered inner dilators; or pericardial entry may be performed via a surgical pericardial window, using a 4 cm long skin incision and soft tissue dissection to form a tract to the inferior border of the pericardial sac. Needle entry during percutaneous pericardial access may be performed under fluoroscopic guidance, while a pericardial window involves grasping and incision of the pericardial membrane under direct vision. A surgical pericardial window approach may be utilized in the event that percutaneous needle entry may be hazardous; for example, if adhesions are present between the pericardium and the myocardium.

A spiral anchor wire can be embedded in the left lateral border of the pericardium prior to insertion of the ventricular assist balloon catheter. The balloon catheter can contain a dedicated guidewire lumen that extends the length of the balloon, with an entry port situated distal to the balloon and an exit port lying proximal to the balloon. After pericardial fixation, the proximal end of the spiral anchor wire can be inserted into the entry port of the guidewire lumen in the ventricular assist balloon catheter, and the balloon catheter can be advanced into position over the myocardium along the anchor wire. The anchor wire can stabilize the tip of the balloon catheter, and it may prevent the balloon from migrating out of position during inflation. The anchor wire can remain in position upon balloon exchange during long term cardiac assistance, also known as destination therapy, and can facilitate placement and proper positioning of the new balloon. The proximal end of the anchor wire can attach to either the subcutaneous air exchange reservoir, or to the subcutaneous anchor block when the air exchange reservoir is not used.

The intrapericardial cardiac assist catheter may be anchored solely at the point of entry in the pericardium. The anchor can comprise a balloon or a wire. The anchor wire can comprise a spiral or helical anchor wire. In some cases, the spiral anchor wire may enter the pericardium near the apex of the heart and be inserted into the lateral aspect of the pericardium. A catheter that contains a dedicated lumen that extends the length of the balloon may be advanced along the wire with the pericardial anchor maintaining the position of the ventricular compression balloon. A small reservoir may be attached to the proximal end of the catheter. One surface of the reservoir may contain a septum or other elastic portion through which a needle may be inserted for balloon inflation, while sealing against the inserted needle. Alternatively, the entire reservoir may be constructed of elastic material that self-seals against needle punctures. Following catheter placement, the reservoir may be implanted subcutaneously via a small skin incision oriented such that the elastic surface faces out towards the skin. The balloon catheter and attached subcutaneous reservoir can remain implanted while an inflation pump, controller and battery reside external to the patient's skin, with a transcutaneous needle used to inflate and deflate the ventricular compression balloon via the implanted reservoir. This configuration can facilitate pump and battery replacement, while decreasing the potential for catheter infection, as a small diameter needle traverses the skin. The needle puncture site may also be changed periodically to decrease the potential for needle tract infection.

In some embodiments, the percutaneous ventricular assist device may comprise a balloon catheter that is inserted into the patient's pericardium near the apex of the heart, via a subxiphoid puncture site. A reservoir containing an elastomeric face can be attached to the proximal end of the catheter. The 4 cm incision can be performed to extend the subxiphoid puncture site, and the reservoir can be implanted subcutaneously in the epigastric region of the abdominal wall. In such devices, a single needle can penetrate the patient's skin and the elastic face of the reservoir to allow the pump unit external to the patient to inflate and deflate the balloon catheter, compressing the patient's ventricle during cardiac systole. Balloon inflation can be triggered by the patient's ECG, as sensed in real time.

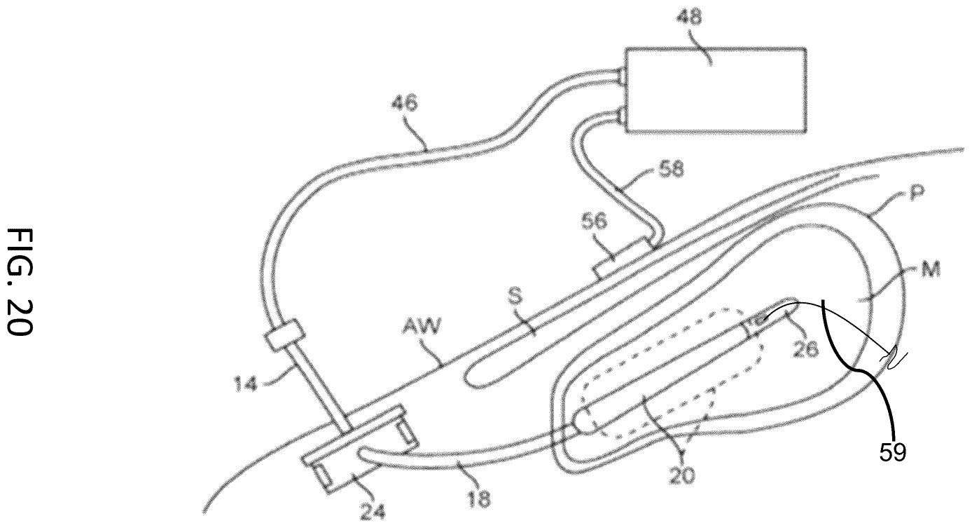

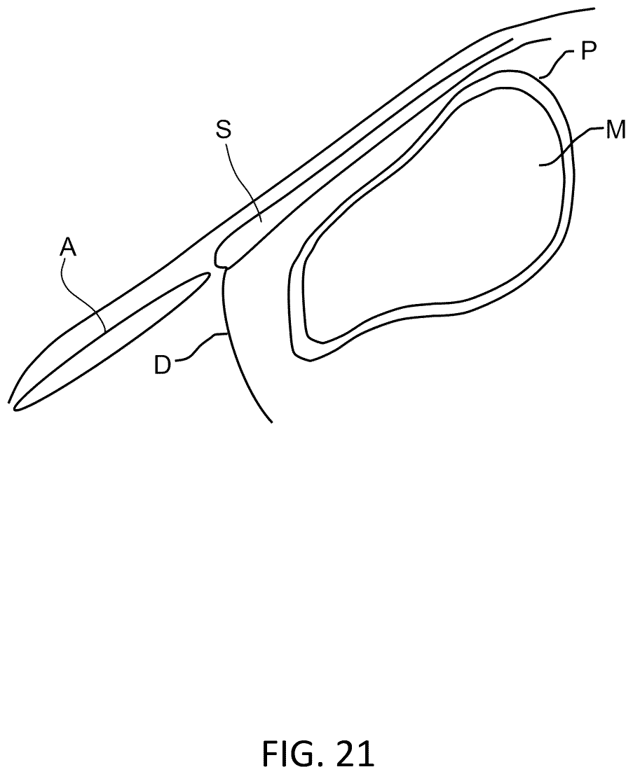

FIG. 21 depicts the anatomical structures encountered during a subxiphoid access to the pericardium P and myocardium M. The xiphoid process is the lowermost portion of the sternum S. The abdominal wall A lies inferior to the sternum S, and the diaphragm D separates the pericardium P and myocardium M from the abdominal cavity.

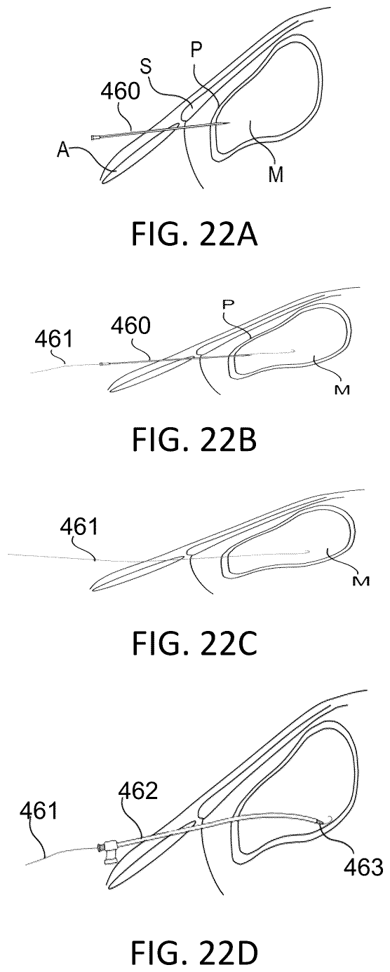

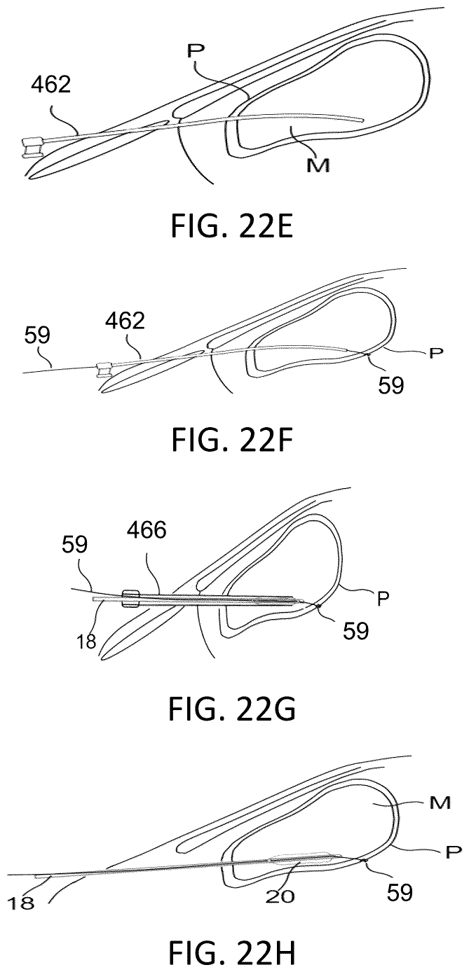

FIGS. 22A-22H depict perspective views of steps used during needle pericardial entry and percutaneous insertion of the cardiac assist system. FIG. 22A illustrates percutaneous entry through the pericardium P, using a long, small bore hypodermic needle 460, which can traverse the abdominal wall A and the diaphragm D in its path to the pericardium P. Following entry through the pericardium P, a guidewire 461 can be inserted into the space between the pericardium P and the myocardium M, as shown in FIG. 22B. The guidewire 461 can be left in position while the needle 460 is removed, as shown in FIG. 22C. FIG. 22D illustrates the next step of advancing an articulating vascular sheath 462 with an inner tapered dilator 463 over the guidewire 461. FIG. 22E illustrates that once in position, the dilator and guidewire can be removed from the patient, leaving the sheath 462 in place between the myocardium M and pericardium P. FIG. 22F shows insertion of the spiral anchor wire 59 through sheath 462 into attachment into a lateral aspect of pericardium P. Once the spiral anchor wire 59 is attached to the pericardium P, the articulating vascular sheath 462 can be removed from the patient. FIG. 22G illustrates insertion of the ventricular assist balloon catheter 18 sheathed in balloon delivery sheath 466, inside the pericardium P. The balloon catheter 18 can be advanced along spiral anchor wire 59. Anchor wire 59 can comprise an elongated wire with a spiral at the distal tip, wherein the distal tip comprises the anchor and the wire component can be used to bring the catheter near the spiral anchor. FIG. 22H shows the deployed ventricular assist balloon catheter 18 anterior to the myocardium M inside the pericardium P stabilized in position by anchor wire 59 which can enter balloon catheter 18 distal to the balloon 20 and can exit the balloon catheter 18 proximal to the balloon 20.

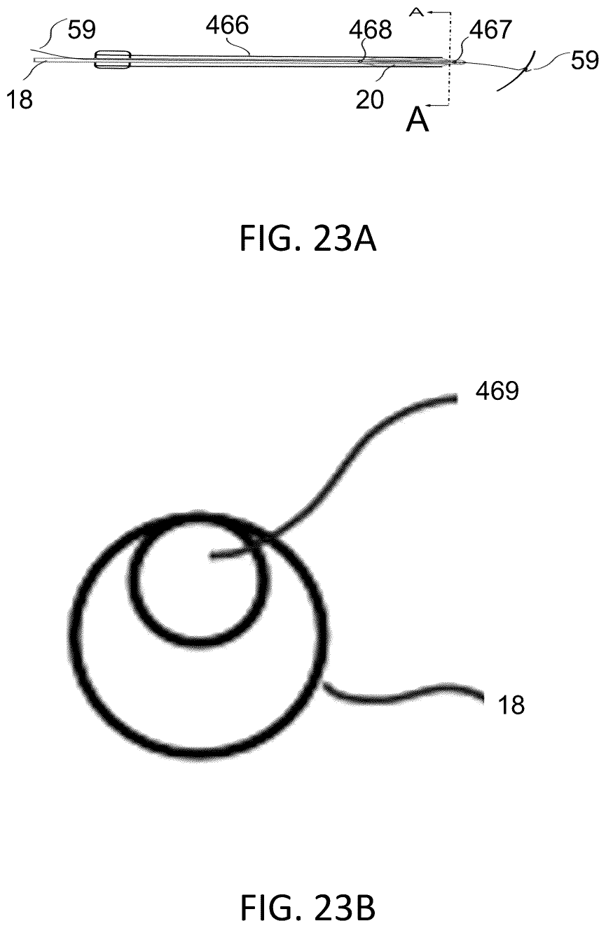

FIGS. 23A-23B show side (23A) and cross-sectional (23B) views of the configuration of the catheter lumen in the cardiac assist system that accommodates the spiral pericardial anchor wire. FIG. 23A shows the configuration of the ventricular assist balloon catheter 18 inside vascular delivery sheath 466 as it is advanced along spiral anchor wire 59. The anchor wire 59 can enter a dedicated lumen in the ventricular assist balloon catheter 18 via port 467 distal to the balloon 20 and exit the balloon catheter 18 via port 468 proximal to the balloon 20. The shaft of anchor wire 59 can lie adjacent to the balloon catheter 18 as both exit the proximal end of balloon delivery sheath 466. FIG. 23B is a cross-section through balloon catheter 18 immediately proximal to port 467, showing dedicated anchor wire lumen 469 lying inside balloon catheter lumen 18.

FIG. 24 illustrates access to the pericardium P via a surgical pericardial window W. A skin incision can be performed at the inferior aspect of the sternum S. Sharp and blunt incision of the underlying soft tissue can be performed to expose the diaphragm D, and the pericardium P lying superior to the diaphragm D. Multiple surgical retractors 470 can be used to retract incised tissue and provide exposure to the pericardium P.

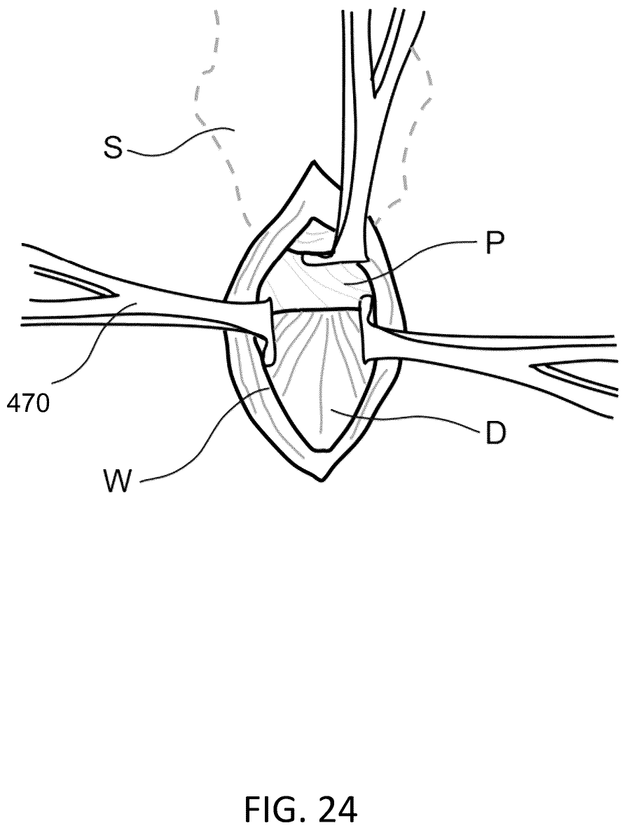

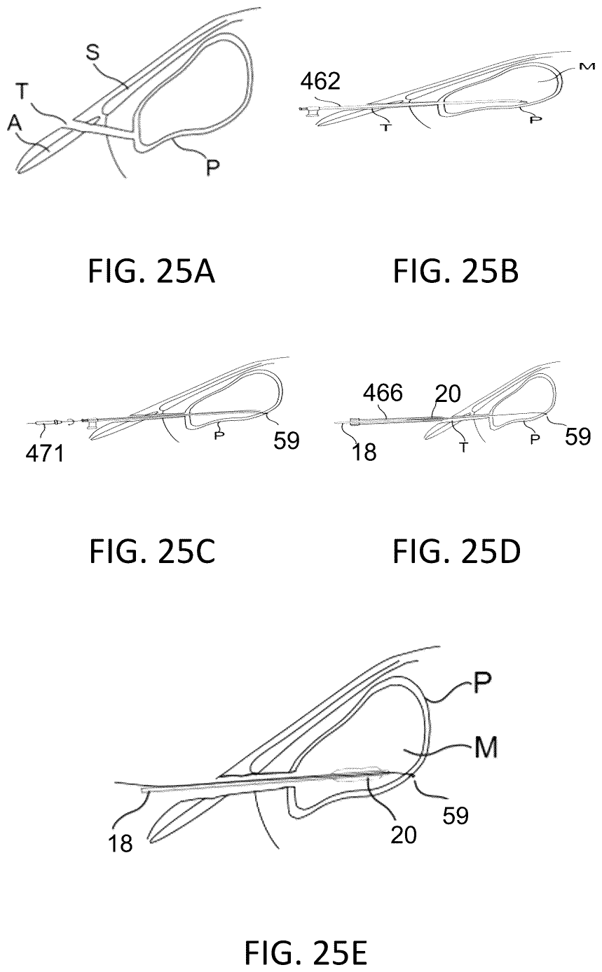

FIGS. 25A-25E depict cross-sectional perspective views of steps used during open surgical pericardial access and insertion of the cardiac assist system into the pericardial sac. FIG. 25A shows a cross-sectional view illustrating anatomical structures associated with a surgical pericardial window access to the pericardium. A surgical tract T can be formed inferior to the sternum S, coursing superiorly through soft tissue including the abdominal wall A, to allow an opening to be formed in the pericardium P. FIG. 25B shows that once surgical access has been achieved, an articulating vascular sheath 462 can be advanced through surgical tract T into pericardial sac P anterior to the myocardium M. FIG. 25C shows advancement of the spiral anchor wire 59 through the vascular sheath 462 into position in the lateral aspect of the pericardium P. Rotation of the spiral anchor wire 59 for permanent fixation can be aided by wire torquer device 471, which is a pin vise tool that grasps the shaft of anchor wire 59 and allows the distal spiral end to be rotated into position in pericardium P. FIG. 25D shows the ventricular assist balloon 20 which is constrained inside a large bore vascular sheath 466 that can be advanced along spiral anchor wire 59 into the opening in the pericardium P. FIG. 25E shows the resting position of the ventricular assist balloon 20 anterior to the myocardium M inside the pericardial sac P, with its positioning maintained by spiral anchor wire 465.

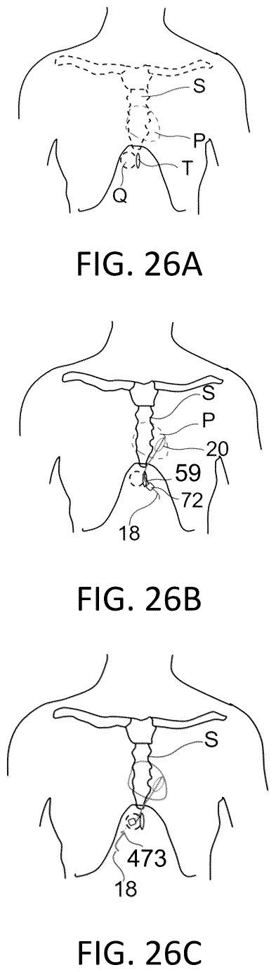

FIGS. 26A-26C illustrate frontal views of stabilization of the catheter body in a subcutaneous pocket prior to exit of the catheter body from the patient. FIG. 26A is a frontal view of the torso of the patient, showing the skin incision for the surgical tract T providing access to the pericardium P. Surgical tract T can be in a subxiphoid location, inferior to the sternum S. A subcutaneous pocket Q can be created using blunt dissection immediately lateral to the skin incision for surgical tract T, to accommodate the proximal end of the ventricular assist balloon catheter. FIG. 26B shows the ventricular assist balloon 20 in position inside the pericardium P, with the proximal length of the ventricular assist balloon catheter 18 and the proximal end of the spiral anchor wire 59 exiting the subxiphoid skin incision and attaching to the anchor block 472. Anchor wire 59 can comprise an elongated wire with a spiral at the distal tip, wherein the distal tip comprises the anchor and the wire component can be used to bring the catheter near the spiral anchor. The pocket can house the subcutaneous reservoir if an implantable reservoir is used and a transcutaneous needle may be used to transmit air from the external pump unit through the skin to inflate the balloon. If a subcutaneous reservoir is not used, an anchor block can be attached to the proximal end of the anchor wire and to the proximal section of the balloon catheter, leaving a length of balloon catheter free to exit the body from a small stab incision in the skin overlying the subcutaneous pocket. The proximal end of the balloon catheter can be connected to the inflation line from the pump external to the patient. FIG. 26C shows the anchor block 472 inserted into the subcutaneous pocket Q. A tiny skin stab incision 473 can be performed to allow the proximal end of the balloon catheter 18 to exit the patient for attachment to the external inflation pump unit.

Although the above steps show methods for implanting a cardiac assist catheter for treating a patient in accordance with embodiments, a person of ordinary skill in the art will recognize many variations based on the teaching described herein. The steps may be completed in a different order. Steps may be added or deleted. Some of the steps may comprise sub-steps. Many of the steps may be repeated as often as beneficial to the procedure.

### Methods of Assisting Cardiac Function

Disclosed herein is a method for assisting cardiac function in a patient suffering from heart failure. The method may comprise detecting the patient's ECG to determine a cardiac rhythm. An implanted port within the patient may be percutaneously accessed with a cannula, and a driving gas may be delivered through the cannula to the port connected then delivered to a pneumatic effector implanted over the patient's left ventricle. Delivery and extraction of the driving gas can be synchronized with a sensed cardiac rhythm to cause the pneumatic effector to compress the heart at a rate which generally matches the patient's natural cardiac rhythm.

In some embodiments, the pneumatic effector can be implanted beneath the patient's pericardial sac and over a myocardial surface. In some embodiments, the pneumatic effector can be on a cardiac assist catheter which enters through the pericardium at an entry location and, in some cases, a channel which may accommodate the shaft of an anchor wire whose distal tip is anchored in the lateral aspect of the pericardium.

In some embodiments, the ECG may be detected in a variety of ways. For example, one or more ECG electrodes may be located on the implanted port, on the implanted pneumatic effector, or elsewhere on the implanted portion of the system. In some cases, a signal from the implanted ECG electrode(s) may be delivered to the ECG circuitry in the external controller using the cannula as a percutaneous transmission element or conductor. In some embodiments, the cannula, or a plurality of cannulas, may themselves act as the ECG electrodes after they can be percutaneously introduced to the implanted port. The ECG electrodes may reside in a subcutaneous location, advanced along the chest of the patient from the subxiphoid skin incision. In some embodiments, the ECG signal may be measured by one or more external electrodes attached to the patients in a conventional manner. Single channel ECG may be used. In some cases, at least two-channel ECG can be employed. Three-channel ECG, four-channel ECG, or greater can be employed. Electrodes may also be placed on the implanted pneumatic effector to provide cardiac pacing in the event of bradycardia in the patient or cardiac defibrillation in the event of ventricular tachycardia, ventricular fibrillation, or asystole in the patient.

The methods of the present disclosure can further comprise detecting an abnormal cardiac rhythm. The abnormal cardiac rhythm can be determined based on the measured ECG, and the presence of an abnormal cardiac rhythm can result in stopping or modifying the operation of the pneumatic effector. For example, the driving gas may be stopped when an abnormal cardiac rhythm is detected. An abnormal rate can be below a predetermined fixed rate. Alternatively, or in addition, a delivery rate of the driving gas may be changed when an abnormal cardiac rhythm is detected. For example, the driving rate may be reduced in the presence of a rapid heartbeat (tachycardia) or increased in the presence of a slow heartbeat (bradycardia).

The methods of the present disclosure can further comprise protocols for treating the patient should the tissue access site become infected. In such cases, the needle or other cannula may be withdrawn from the tissue tract, the tissue tract treated for infection, and the cannula (either disinfected or a new cannula) reintroduced to the implanted port; in some cases, through a different access route. For example, for septum-type ports, the cannula may be introduced to a different region of the part through a new tissue tract in non-infected tissue.

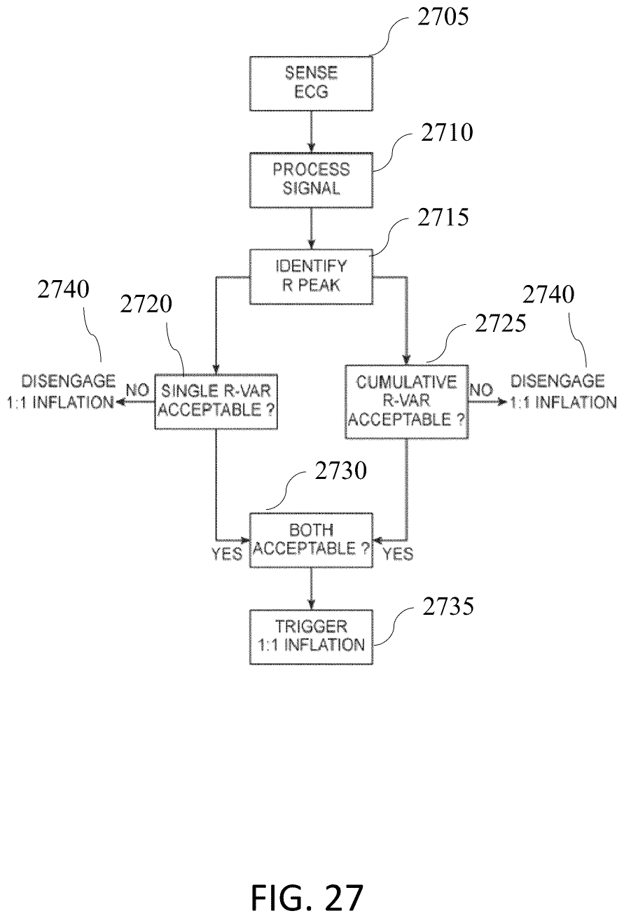

Referring now to FIG. 27, an exemplary protocol 2700 for operating the pneumatic effectors of the present disclosure is described. The patient's ECG can be measured (in a step 2705) using any of the ECG electrodes described previously. The ECG measurement circuitry may be incorporated into the external drive unit and can operate on ECG measurement principles. The raw ECG principles may undergo processing (in a step 2710), for example digital processing for removing motion artifacts from the signal, and then the processed signal can be scanned to determine the occurrence of signal artifacts associated with the patient cardiac rhythm, which can be through measuring R peaks (in a step 2715).

While the R peaks can be used to directly drive the pump assembly and pneumatic effector, the R peak pattern can be evaluated to determine if it is normal or abnormal. For example, the occurrence of successive single peak R values may be compared (in a step 2720) to determine whether they can be increasing or decreasing in length. If the R-R peak interval remains constant within ±10 percent of the previous interval, cardiac rhythm can be considered normal and the trigger may be generated (in a step 2735). A second abnormality test can be applied to the R-R interval over a cumulative number of beats (in a step 2725), for example between about 5-15 beats. If an R-R interval is larger than a threshold amount, for example between about 5 -15 percent, of the mean R-R interval of the preceding about 5-15 heart beats, the cardiac rhythm is considered abnormal. A determination of normality by both abnormality tests (in a step 2730) can trigger inflation (in the step 2735).

In the event an abnormal cardiac rhythm is detected, the system may take any one of a number of actions. For example, the system may shut down triggering of the pneumatic effector (in a steps 2740) until the patient's native cardiac rhythm returns to normal. Alternatively, in the case of rapid heartbeat, the 1:1 synchronization between the natural heart rhythm and the triggering of the pneumatic effector may be altered, for example the effector may be triggered on every second natural beat (a 2:1 ratio), every third beat (a 3:1 ratio), more often, less often, or the like. Actuation of the pneumatic effector at a 1:1 ratio may be resumed as soon as the cardiac rhythm returns to normal. In some cases, actuation of the pneumatic effector at a 1:1 ratio may be resumed before the cardiac rhythm returns to normal or after the cardiac rhythm returns to normal.

Although the above steps show methods for assisting cardiac function and process 2700 for treating a patient in accordance with embodiments, a person of ordinary skill in the art will recognize many variations based on the teaching described herein. The steps may be completed in a different order. Steps may be added or deleted. Some of the steps may comprise sub-steps. Many of the steps may be repeated as often as beneficial to the procedure.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers, and also represent an amount close to the stated amount that still performs a desired function or achieves a desired result. The term “about” or “approximately” may mean within an acceptable error range for the particular value, which will depend in part on how the value is measured or determined, e.g., the limitations of the measurement system. For example, the terms “approximately”, “about”, and “substantially” may refer to an amount that is within less than 10% of, within less than 5% of, within less than 1% of, within less than 0.1% of, and within less than 0.01% of the stated amount. For example, “about” may mean within 1 or more than 1 standard deviation, per the practice in the art. Alternatively, “about” may mean a range of up to 20%, up to 10%, up to 5%, or up to 1% of a given value. As used herein, the term “about” a number refers to that number plus or minus 10% of that number. The term “about” a range refers to that range minus 10% of its lowest value and plus 10% of its greatest value. Where particular values can be described in the application and claims, unless otherwise stated the term “about” meaning within an acceptable error range for the particular value may be assumed.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” can be often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” can be often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “ex vivo” is used to describe an event that takes place outside of a subject's body. An ex vivo assay is not performed on a subject. Rather, it is performed upon a sample separate from a subject.

As used herein, the terms “treatment” or “treating” can be used in reference to a pharmaceutical or other intervention regimen for obtaining beneficial or desired results in the recipient. Beneficial or desired results include but may not be limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and should not be construed as limiting the subject matter described.

### EXAMPLES

The following examples are included for illustrative purposes only and are not be intended to limit the scope of the present disclosure.

### Example 1: Ex-Vivo Testing of Balloon Ventricular Compression

Ex-vivo testing of ventricular compression using differently sized pneumatic effector balloons was done using fresh explanted porcine hearts with an intact pericardium. A 75 cc volume balloon achieved only a modest cardiac output, measured via a digital flow transducer at the aortic outlet. When the balloon volume was increased to 120 cc, balloon inflation at 60 bpm yielded a cardiac output of 100 ml/min, equivalent to 50% of the baseline cardiac output achieved via open heart massage at 60 bpm.

### Example 2: Implantation of a Cardiac Assist Device

A patient with cardiac abnormalities who would benefit from a ventricular assist device enters a hospital to undergo a procedure. During the procedure, the physician inserts an implantable port fluidically coupled to a pneumatic effector. The physician follows the following steps:
1. Insert a hypodermic needle through the abdominal wall and the diaphragm and into the pericardium
2. Advance a guide wire into the space between the pericardium and the myocardium
3. Remove the needle
4. Advance deflectable and/or articulating sheath with a dilator (e.g., inner tapered dilator) over the guidewire
5. Remove the guide wire and dilator, leaving the sheath inserted
6. Advance the anchoring device through the sheath into the lateral aspect of the pericardium
7. Once in contact with the pericardium, rotate the anchor device via a torque device to implant into pericardium
8. Lightly tug the anchor device to make sure it is secured with the pericardium
9. Remove the sheath
10. Advance a balloon catheter sheathed in a balloon delivery sheath over anchor device wire until the balloon catheter is advanced to the distal point
11. Insert the proximal end of the anchor device into an anchor block
12. Use the torque driver to secure the block on the proximal end of the device
13. Create a subcutaneous pocket in a subxiphoid location, inferior to the sternum
14. Insert the balloon catheter, anchor device, and block into the pocket
15. Make a small incision at the proximal end of the pocket to expose the balloon catheter
16. Attach balloon catheter to the leak detector device
17. Attach the leak detection device to the driveline
18. Create a tunnel next to the sternum
19. Advance ECG catheter inside the tunnel
20. Connect the ECG catheter to the control box
21. Activate the control box to inflate/deflate the balloon

The pneumatic effector is guided towards the pericardium to lie between the myocardium and the pericardial sac anterior to the left ventricle. The pneumatic effector is anchored there by an anchor. Once implanted, a pump controlled by circuitry can drive a fluid, for example a gas, into the pneumatic effector for it to expand and contract. These rhythmic expansions and contractions can mimic the beat of the heart and help the heart to function at a proper rhythm. If the heart deviates from a proper rhythm, the circuitry can change the rhythm of the pump to account for the change in rhythm.

### EMBODIMENTS

Embodiment 1. An implantable cardiac assist system, comprising: (a) an external drive unit; (b) an anchor wire configured to be anchored to a pericardium of a subject; (c) an implantable cardiac assist catheter comprising a distal section; and (d) a pneumatic effector configured to be operatively coupled to the external drive unit, wherein the pneumatic effector is coupled to the distal section of the implantable cardiac assist catheter and configured to be implanted beneath a pericardial sac of the subject and over a myocardial surface overlying a left ventricle of the subject; wherein the distal section of the implantable cardiac assist catheter has a guidewire lumen with a first port proximal to the pneumatic effector and a second port distal to the pneumatic effector, and wherein the implantable cardiac assist catheter is configured to be advanced over the anchor wire via the guidewire lumen.

Embodiment 2. The cardiac assist system of embodiment 1, wherein the external drive unit comprises (a) a pump assembly; and (b) control circuitry configured to operate the pump to actuate the pneumatic effector in response to the patient's sensed heart rhythm.

Embodiment 3. The cardiac assist system of embodiment 1 or 2, further comprising an implantable port configured to receive a percutaneously introduced cannula, wherein the implanted port is connected to supply a driving gas received from the cannula to the pneumatic effector.

Embodiment 4. The cardiac assist system of embodiment 3, further comprising, a connecting tube having a pump end attachable to the pump assembly and a cannula end attached to the cannula.

Embodiment 5. The cardiac assist system of any one of embodiments 1 to 4, wherein the anchor wire is configured to anchor the pneumatic effector to a position anterior to the heart.

Embodiment 6. The cardiac assist system of any one of embodiments 1-5, wherein the anchor wire comprises a spiral anchor positioned at a distal tip of the anchor wire.

Embodiment 7. The cardiac assist system of embodiment 6, wherein the spiral anchor is configured to be rotated and advanced into the pericardium of the subject to anchor it into the pericardium.

Embodiment 8. The cardiac assist system of embodiment 6 or 7, wherein the guidewire lumen is configured to suppress an axial motion of the pneumatic effector along the anchor wire disposed inside the guidewire lumen to substantially eliminate traction on the spiral anchor when the spiral anchor is embedded in the pericardium.

Embodiment 9. A method for implanting a cardiac assist system, the method comprising: (a) inserting a guidewire into an intrapericardial space anterior to a heart of a subject at an apical site; (b) advancing a vascular sheath and a tapered dilator over the guidewire; (c) advancing a pericardial anchor wire to a lateral aspect of a pericardium of the heart; (d) anchoring the pericardial anchor wire to the lateral aspect of the pericardium; and (e) advancing an implantable cardiac assist catheter over the anchor wire via a guidewire lumen of the implantable cardiac assist catheter with a first port proximal to the pneumatic effector and a second port distal to the pneumatic effector, thereby positioning the implantable cardiac assist catheter anterior to the heart.

Embodiment 10. The method of embodiment 9, wherein anchoring a pericardial anchor wire comprises rotating and advancing a spiral anchor disposed on a distal end of the pericardial anchor wire into the pericardium.

Embodiment 11. The method of embodiment 9 or 10, wherein the pneumatic effector is configured to be implanted beneath a pericardial sac of the subject and over a myocardial surface overlying a left ventricle of the subject.

Embodiment 12. The method of any one of embodiments embodiment 9 to 11, further comprising creating a subcutaneous pocked in a subxiphoid location inferior to the sternum, wherein a part of the pericardial anchor wire and the implantable cardiac assist catheter are inserted into the subcutaneous pocket.

Embodiment 13. The method of any one of embodiments 9 to 12, further comprising coupling the pneumatic effector to a leak detector device.

Embodiment 14. The method of any one of embodiments 9 to 13, further comprising electrically coupling the pneumatic effector to an ECG control box.

Embodiment 15. A cardiac assist system, comprising: (a) an external drive unit; (b) an implantable cardiac assist catheter comprising a distal section having a guidewire lumen; (c) a pneumatic effector configured to be operatively coupled to the external drive unit, wherein the pneumatic effector is coupled to the distal section of the implantable cardiac assist catheter, wherein the distal section of the implantable cardiac assist catheter has a first port proximal to the pneumatic effector and a second port distal to the pneumatic effector, wherein the pneumatic effector is configured to be implanted beneath a pericardial sac of the subject and over a myocardial surface overlying a left ventricle of the subject, and wherein the pneumatic effector comprises a partially elastic balloon configured to inflate with a cushioning effect on a myocardium of the subject; and (d) an anchor wire, wherein the anchor wire is configured to anchor the partially elastic balloon to a position anterior to the heart.

Embodiment 16. The cardiac assist system of embodiment 15, wherein the distal section of the implantable cardiac assist catheter has a first port proximal to the pneumatic effector and a second port distal to the pneumatic effector.

Embodiment 17. The cardiac assist system of embodiment 15 or 16, wherein the external drive unit comprises (a) a pump assembly; and (b) control circuitry configured to operate the pump to actuate the pneumatic effector in response to the patient's sensed heart rhythm.

Embodiment 18. The cardiac assist system of any one of embodiments 15 to 17, further comprising an implantable port configured to receive a percutaneously introduced cannula, wherein the implanted port is connected to supply a driving gas received from the cannula to the pneumatic effector.

Embodiment 19. The cardiac assist system of embodiment 18, further comprising, a connecting tube having a pump end attachable to the pump assembly and a cannula end attached to the cannula.

Embodiment 20. The cardiac assist system of any one of embodiments 15 to 19, wherein the anchor wire is configured to anchor the partially elastic balloon to a position anterior to the heart.

Embodiment 21. The cardiac assist system of any one of embodiments 15 to 20, wherein the anchor wire comprises a spiral anchor at a distal tip of the anchor wire.

Embodiment 22. The cardiac assist system of embodiment 21, wherein the spiral anchor is configured to be torqued into the pericardium of the subject to anchor it into the pericardium.

Embodiment 23. The cardiac assist system of any one of embodiments 15 to 22, wherein the partially elastic balloon comprises polyurethane.

Embodiment 24. The cardiac assist system of any one of embodiments 15 to 23, wherein the partially elastic balloon has at least one fatigue property such that the partially elastic balloon is usable as anchored to the position anterior to the heart for at least one year without being exchanged.

Embodiment 25. The cardiac assist system of any one of embodiments 15 to 24, wherein the partially elastic balloon is configured to partially yield to the pressure on the partially elastic balloon from the heart.

Embodiment 26. The cardiac assist system of any one of embodiments 15 to 25, wherein the partially elastic balloon has an elastic modulus of from 4.7 MPa to 7.4 MPa.

Embodiment 27. A method for implanting a cardiac assist system, the method comprising: (a) anchoring a pericardial anchor wire to a lateral aspect of a pericardium; (b) advancing a guidewire lumen of an implantable cardiac assist catheter over the anchor wire into a position anterior to a heart; and (c) anchoring a pneumatic effector to the position anterior to the heart, the pneumatic effector being coupled to a distal end of the implantable cardiac assist catheter and configured to be implanted beneath a pericardial sac of the subject and over a myocardial surface overlying a left ventricle of the subject, wherein the pneumatic effector comprises a partially elastic balloon; wherein the partially elastic balloon is configured to inflate with a cushioning effect on a myocardium of the subject at the position anterior to the heart in response to gas being driven thereto.

Embodiment 28. The method of embodiment 27, wherein the partially elastic balloon comprises polyurethane.

Embodiment 29. The method of embodiment 27 or 28, further comprising repeatedly expanding and contracting the partially elastic balloon anchored to the position anterior to the heart for at least one year without exchanging said partially elastic balloon, wherein the partially elastic balloon has at least one improved fatigue property such that said partially elastic balloon is usable for at least one year without being exchanged.

Embodiment 30. The method of any one of embodiments 27 to 29, wherein the partially elastic balloon partially yields to the pressure on the partially elastic balloon from the heart.

Embodiment 31. The method of any one of embodiments 27 to 30, wherein the partially elastic balloon has an elastic modulus of from 4.7 MPa to 7.4 MPa.

Embodiment 32. The method of any one of embodiments 27 to 31, wherein anchoring a pericardial anchor wire comprises rotating and advancing a spiral anchor disposed on a distal end of the pericardial anchor wire into the pericardium.

Embodiment 33. A method for assisting cardiac function in a subject suffering from heart failure, the method comprising: (a) measuring the subject's ECG to determine a cardiac rhythm; and (b) delivering a driving gas to expand and contract a pneumatic effector implanted over the subject's left ventricle in response to the measured ECG, wherein the pneumatic effector comprises a partially elastic balloon; wherein the driving gas delivery is synchronized with the determined cardiac rhythm to cause the partially elastic balloon to compress a heart of the subject at a rate which matches the cardiac rhythm, wherein the partially elastic balloon is configured to inflate with a cushioning effect on a myocardium of the subject at a position anterior to the heart in response to gas being driven thereto.

Embodiment 34. The method of embodiment 33, wherein delivering driving gas comprises percutaneously accessing an implanted port connected to the pneumatic effector with a cannula through which the driving gas is delivered.

Embodiment 35. The method of embodiment 33 or 34, wherein the partially elastic balloon comprises polyurethane.

Embodiment 36. The method of any one of embodiments 33 to 35, further comprising repeatedly expanding and contracting the partially elastic balloon implanted over the subject's left ventricle for at least one year without exchanging said partially elastic balloon, wherein the partially elastic balloon has at least one improved fatigue property such that said partially elastic balloon is usable used for at least one year without being exchanged.

Embodiment 37. The method of any one of embodiments 33 to 36, wherein the partially elastic balloon partially yields to the pressure on the partially elastic balloon from the heart.

Embodiment 38. The method of any one of embodiments 33 to 37, wherein the partially elastic balloon has an elastic modulus of from 4.7 MPa to 7.4 MPa.

Embodiment 39. The method of any one of embodiments 33 to 38, wherein the pneumatic effector is implanted beneath the patient's pericardial sac and over a myocardial surface.

Embodiment 40. The method of any one of embodiments 33 to 39, wherein the ECG is measured with one or more electrodes located on an implanted pneumatic port.

Embodiment 41. The method of any one of embodiments 33 to 40, wherein the ECG is measured with one or more electrodes located on the implanted pneumatic effector.

Embodiment 42. The method of any one of embodiments 33 to 41, wherein the ECG is measured with a cannula acting as an ECG electrode.

Embodiment 43. The method of any one of embodiments 33 to 42, wherein the ECG is measured with an external electrode.

Embodiment 44. The method of any one of embodiments 33 to 43, wherein the ECG is measured with an ECG electrode located subcutaneously.

Embodiment 45. The method of any one of embodiments 33 to 44, further comprising detecting an abnormal cardiac rhythm.

Embodiment 46. The method of embodiment 45, wherein delivery of the driving gas is stopped when an abnormal cardiac rhythm is detected.

Embodiment 47. The method of embodiment 45 or 46, wherein a rate of delivering the driving gas is changed when an abnormal cardiac rhythm is detected.

Embodiment 48. The method of embodiment 47, wherein the rate is below a rate of the detected cardiac rhythm.

Embodiment The method of embodiment 47 or 48, wherein the rate is a predetermined fixed rate.

Embodiment 50. The method of any one of embodiments 33 to 49, further comprising removing the cannula from the access site when an infection of the access site is observed.

Embodiment 51. The method of embodiment 50, further comprising treating the infection and replacing the cannula in the implanted port.

Embodiment 52. The method of embodiment 50 or 51, wherein the cannula is replaced in through a different site than enters the port through a different location.

Embodiment 53. The method of embodiment 52, wherein the cannula comprises a needle and the port comprise a needle-penetrable penetrable septum and the different location is a different region on the septum.

Embodiment 54. A cardiac assist system comprising: (a) an external drive unit; (b) an implantable cardiac assist catheter comprising a distal section; (c) a pneumatic effector configured to be operatively coupled to the external drive unit, wherein the pneumatic effector is coupled to the distal section of the implantable cardiac assist catheter and configured to be implanted beneath a pericardial sac of the subject and over a myocardial surface overlying a left ventricle of the subject, and wherein the pneumatic effector comprises a balloon having a size of at least about 120 cc and configured to displace a left ventricular wall by at least about 4 cm.

Embodiment 55. The cardiac assist system of embodiment 54, wherein the distal section of the implantable cardiac assist catheter has a guidewire lumen with a first port proximal to the pneumatic effector and a second port distal to the pneumatic effector.

Embodiment 56. The cardiac assist system of embodiment 54 or 55, wherein the external drive unit comprises (a) a pump assembly; and (b) control circuitry configured to operate the pump to actuate the pneumatic effector in response to the patient's sensed heart rhythm.

Embodiment 57. The cardiac assist system of any one of embodiments 54 to 56, further comprising an implantable port configured to receive a percutaneously introduced cannula, wherein the implanted port is connected to supply a driving gas received from the cannula to the pneumatic effector.

Embodiment 58. The cardiac assist system of embodiment 57, further comprising, a connecting tube having a pump end attachable to the pump assembly and a cannula end attached to the cannula.

Embodiment 59. The cardiac assist system of any one of embodiments 54 to 58, further comprising an anchor wire, wherein the anchor wire is configured to anchor the balloon to a position anterior to the heart.

Embodiment 60. The cardiac assist system of any one of embodiments 54 to 59, wherein the anchor wire is configured to anchor the balloon to a position anterior to the heart.

Embodiment 61. The cardiac assist system of embodiment 60, wherein the anchor wire comprises a spiral anchor at a distal tip of the anchor wire.

Embodiment 62. The cardiac assist system of embodiment 61, wherein the spiral anchor is configured to be rotated and advanced into the pericardium of the subject to anchor it into the pericardium.

Embodiment 63. The cardiac assist system of any one of embodiments 54 to 62, wherein the balloon is configured to generate a cardiac output of about 100 ml/min.

Embodiment 64. The cardiac assist system of any one of embodiments 54 to 63, wherein the balloon is configured to displace a left ventricular wall by at least about 5 cm.

Embodiment 65. The cardiac assist system of any one of embodiments 54 to 64, wherein the balloon is configured to generate a cardiac output approximately equivalent to 50% of the baseline cardiac output achieved via open heart massage at 60 bpm.

Embodiment 66. A method for implanting a cardiac assist system, the method comprising: (a) anchoring a pericardial anchor wire to a lateral aspect of a pericardium; (b) advancing an implantable cardiac assist catheter into a position anterior to a heart; and (c) anchoring a pneumatic effector to the position anterior to the heart, the pneumatic effector being coupled to a distal end of the implantable cardiac assist catheter, wherein the pneumatic effector comprises a balloon having a size of at least about 120 cc; wherein the partially elastic balloon is configured to receive a driving gas delivered thereto to be inflated; and wherein the partially elastic balloon is configured to displace a left ventricular wall of the heart by at least about 4 cm in response to being inflated.

Embodiment 67. The method of embodiment 66, wherein the pneumatic effector is configured to be implanted beneath a pericardial sac of the subject and over a myocardial surface overlying a left ventricle of the subject.

Embodiment 68. The method of embodiment 66 or 67, wherein advancing the implantable cardiac assist catheter comprises advancing a guidewire lumen of an implantable cardiac assist catheter over the anchor wire into the position anterior to the heart.

Embodiment 69. The method of any one of embodiments 66 to 68, wherein the balloon is configured to generate a cardiac output of about 100 ml/min.

Embodiment 70. The method of any one of embodiments 66 to 69, wherein the balloon is configured to displace a left ventricular wall by at least about 5 cm.

Embodiment 71. The method of any one of embodiments 66 to 70, wherein the balloon is configured to generate a cardiac output approximately equivalent to 50% of the baseline cardiac output achieved via open heart massage at 60 bpm.

Embodiment 72. The method of any one of embodiments 66 to 71, wherein anchoring a pericardial anchor wire comprises advancing and rotating a spiral anchor disposed on a distal end of the pericardial anchor wire into the pericardium.

Embodiment 73. A method for assisting cardiac function in a subject suffering from heart failure, the method comprising: (a) measuring the subject's ECG to determine a cardiac rhythm; and (b) delivering a driving gas to expand and contract a pneumatic effector implanted over the subject's left ventricle in response to the measured ECG, wherein the pneumatic effector comprises a balloon having a size of at least about 120 cc; wherein the balloon is configured to displace a left ventricular wall by at least about 4 cm while expanding the balloon, wherein the driving gas delivery is synchronized with the determined cardiac rhythm to cause the balloon to compress a heart of the subject at a rate which matches the cardiac rhythm.

Embodiment 74. The method of embodiment 73, wherein delivering driving gas comprises percutaneously accessing an implanted port connected to the pneumatic effector with a cannula through which the driving gas is delivered.

Embodiment 75. The method of embodiment 73 or 74, wherein the pneumatic effector is implanted beneath the patient's pericardial sac and over a myocardial surface.

Embodiment 76. The method of any one of embodiments 73 to 75, wherein the ECG is measured with one or more electrodes located on an implanted pneumatic port.

Embodiment 77. The method of any one of embodiments 73 to 76, wherein the ECG is measured with one or more electrodes located on the implanted pneumatic effector.

Embodiment 78. The method of any one of embodiments 73 to 77, wherein the ECG is measured with a cannula acting as an ECG electrode.

Embodiment 79. The method of any one of embodiments 73 to 78, wherein the ECG is measured with an external electrode.

Embodiment 80. The method of any one of embodiments 73 to 79, wherein the ECG is measured with an ECG electrode located subcutaneously.

Embodiment 81. The method of any one of embodiments 73 to 80, further comprising detecting an abnormal cardiac rhythm.

Embodiment 82. The method of embodiment 81, wherein delivery of the driving gas is stopped when an abnormal cardiac rhythm is detected.

Embodiment 83. The method of embodiment 81 or 82, wherein a rate of delivering the driving gas is changed when an abnormal cardiac rhythm is detected.

Embodiment 84. The method of embodiment 83, wherein the rate is below a rate of the detected cardiac rhythm.

Embodiment 85. The method of embodiment 83 or 84, wherein the rate is a predetermined fixed rate.

Embodiment 86. The method of any one of embodiments 73 to 85, further comprising removing the cannula from the access site when an infection of the access site is observed.

Embodiment 87. The method of embodiment 86, further comprising treating the infection and replacing the cannula in the implanted port.

Embodiment 88. The method of embodiment 86 or 87, wherein the cannula is replaced in through a different site than enters the port through a different location.

Embodiment 89. The method of embodiment 88, wherein the cannula comprises a needle and the port comprises a needle-penetrable penetrable septum and the different location is a different region on the septum.

Embodiment 90. The method of any one of embodiments 73 to 89, wherein the balloon is configured to generate a cardiac output of about 100 ml/min.

Embodiment 91. The method of any one of embodiments 73 to 90, wherein the balloon is configured to displace a left ventricular wall by at least about 5 cm.

Embodiment 92. The method of any one of embodiments 73 to 91, wherein the balloon is configured to generate a cardiac output approximately equivalent to 50% of the baseline cardiac output achieved via open heart massage at 60 bpm.

Embodiment 93. An implantable cardiac assist system, comprising: (a) an external drive unit; (b) an anchor wire configured to be anchored to a pericardium of a subject; (c) an implantable cardiac assist catheter comprising a distal section; and (d) a pneumatic effector configured to be operatively coupled to the external drive unit, wherein the pneumatic effector is coupled to the distal section of the implantable cardiac assist catheter and configured to be implanted beneath a pericardial sac of the subject and over a myocardial surface overlying a left ventricle of the subject, wherein the implantable cardiac assist catheter is configured to be advanced over the anchor wire via the guidewire lumen, and wherein one or more of (i) the pneumatic effector comprises a balloon having a size of at least about 120 cc, wherein the balloon is configured to displace a left ventricular wall by at least about 4 cm while expanding the balloon; (ii) the pneumatic effector comprises a partially elastic balloon configured to inflate with a cushioning effect on a myocardium of the subject at a position anterior to the heart in response to gas being driven thereto; or (iii) the distal section of the implantable cardiac assist catheter has a guidewire lumen with a first port proximal to the pneumatic effector and a second port distal to the pneumatic effector.

Embodiment 94. A method for implanting a cardiac assist system, the method comprising: (a) anchoring a pericardial anchor wire to a lateral aspect of a pericardium; (b) advancing a guidewire lumen of an implantable cardiac assist catheter over the anchor wire into a position anterior to a heart; and (c) anchoring a pneumatic effector to the position anterior to the heart, the pneumatic effector being coupled to a distal end of the implantable cardiac assist catheter and configured to be implanted beneath a pericardial sac of the subject and over a myocardial surface overlying a left ventricle of the subject; wherein one or more of (i) the pneumatic effector comprises a balloon having a size of at least about 120 cc, wherein the balloon is configured to displace a left ventricular wall by at least about 4 cm while expanding the balloon; (ii) the pneumatic effector comprises a partially elastic balloon configured to inflate with a cushioning effect on a myocardium of the subject at a position anterior to the heart in response to gas being driven thereto; or (iii) the distal section of the implantable cardiac assist catheter has a guidewire lumen with a first port proximal to the pneumatic effector and a second port distal to the pneumatic effector.

While preferred embodiments of the present disclosure have been shown and described herein, it will be obvious to those skilled in the art that such embodiments are provided by way of example only. Numerous variations, changes, and substitutions can occur to those skilled in the art without departing from the present disclosure. It should be understood that various alternatives to the embodiments of the present disclosure described herein may be employed in practicing the present disclosure. It is intended that the following claims define the scope of the invention and that methods and structures within the scope of these claims and their equivalents be covered thereby.

1. An implantable cardiac assist system, comprising:
(a) an external drive unit;
(b) an anchor wire configured to be anchored to a pericardium of a subject;
(c) an implantable cardiac assist catheter comprising a distal section; and
(d) a pneumatic effector configured to be operatively coupled to the external drive unit, wherein the pneumatic effector is coupled to the distal section of the implantable cardiac assist catheter and configured to be implanted beneath a pericardial sac of the subject and over a myocardial surface overlying a left ventricle of the subject;
(e) wherein the distal section of the implantable cardiac assist catheter has a guidewire lumen with a first port proximal to the pneumatic effector and a second port distal to the pneumatic effector, and wherein the implantable cardiac assist catheter is configured to be advanced over the anchor wire via the guidewire lumen.

2. The implantable cardiac assist system of claim 1, wherein the external drive unit comprises (a) a pump assembly; and (b) control circuitry configured to operate the pump assembly to actuate the pneumatic effector in response to a patient's sensed heart rhythm.

3. The implantable cardiac assist system of claim 1, further comprising an implantable port configured to receive a percutaneously introduced cannula, wherein the implantable port is connected to supply a driving gas received from the percutaneously introduced cannula to the pneumatic effector.

4. The implantable cardiac assist system of claim 3, further comprising, a connecting tube having a pump end attachable to a pump assembly and a cannula end attached to the percutaneously introduced cannula.

5. The implantable cardiac assist system of claim 1, wherein the anchor wire is configured to anchor the pneumatic effector to a position anterior to a heart.

6. The implantable cardiac assist system of claim 1, wherein the anchor wire comprises a spiral anchor positioned at a distal tip of the anchor wire.

7. The implantable cardiac assist system of claim 6, wherein the spiral anchor is configured to be rotated and advanced into the pericardium of the subject to anchor it into the pericardium.

8. The implantable cardiac assist system of claim 6, wherein the guidewire lumen is configured to suppress an axial motion of the pneumatic effector along the anchor wire disposed inside the guidewire lumen to substantially eliminate traction on the spiral anchor when the spiral anchor is embedded in the pericardium.

9. A method for implanting a cardiac assist system, the method comprising:
(a) inserting a guidewire into an intrapericardial space anterior to a heart of a subject at an apical site;
(b) advancing a vascular sheath and a tapered dilator over the guidewire;
(c) advancing a pericardial anchor wire to a lateral aspect of a pericardium of the heart;
(d) anchoring the pericardial anchor wire to the lateral aspect of the pericardium; and
(e) advancing an implantable cardiac assist catheter over the pericardial anchor wire via a guidewire lumen of the implantable cardiac assist catheter with a first port proximal to a pneumatic effector and a second port distal to the pneumatic effector, thereby positioning the implantable cardiac assist catheter anterior to the heart.

10. The method of claim 9, wherein anchoring the pericardial anchor wire comprises rotating and advancing a spiral anchor disposed on a distal end of the pericardial anchor wire into the pericardium.

11. The method of claim 9, wherein the pneumatic effector is configured to be implanted beneath a pericardial sac of the subject and over a myocardial surface overlying a left ventricle of the subject.

12. The method of claim 9, further comprising creating a subcutaneous pocked in a subxiphoid location inferior to a sternum, wherein a part of the pericardial anchor wire and the implantable cardiac assist catheter are inserted into a subcutaneous pocket.

13. The method of claim 9, further comprising coupling the pneumatic effector to a leak detector device.

14. The method of claim 9, further comprising electrically coupling the pneumatic effector to an ECG control box.

15. A cardiac assist system, comprising:
(a) an external drive unit;
(b) an implantable cardiac assist catheter comprising a distal section having a guidewire lumen;
(c) a pneumatic effector configured to be operatively coupled to the external drive unit, wherein the pneumatic effector is coupled to the distal section of the implantable cardiac assist catheter, wherein the distal section of the implantable cardiac assist catheter has a first port proximal to the pneumatic effector and a second port distal to the pneumatic effector, wherein the pneumatic effector is configured to be implanted beneath a pericardial sac of a subject and over a myocardial surface overlying a left ventricle of the subject, and wherein the pneumatic effector comprises a partially elastic balloon configured to inflate with a cushioning effect on a myocardium of the subject; and
(d) an anchor wire, wherein the anchor wire is configured to anchor the partially elastic balloon to a position anterior to a heart.

16. The cardiac assist system of claim 15, wherein the distal section of the implantable cardiac assist catheter has a first port proximal to the pneumatic effector and a second port distal to the pneumatic effector.

17. The cardiac assist system of claim 15, wherein the external drive unit comprises (a) a pump assembly; and (b) control circuitry configured to operate the pump assembly to actuate the pneumatic effector in response to a patient's sensed heart rhythm.

18. The cardiac assist system of claim 15, further comprising an implantable port configured to receive a percutaneously introduced cannula, wherein the implantable port is connected to supply a driving gas received from the percutaneously introduced cannula to the pneumatic effector.

19. The cardiac assist system of claim 18, further comprising, a connecting tube having a pump end attachable to a pump assembly and a cannula end attached to the percutaneously introduced cannula.

20. The cardiac assist system of claim 15, wherein the anchor wire is configured to anchor the partially elastic balloon to a position anterior to the heart.

21. The cardiac assist system of claim 15, wherein the anchor wire comprises a spiral anchor at a distal tip of the anchor wire.

22. The cardiac assist system of claim 21, wherein the spiral anchor is configured to be torqued into a pericardium of the subject to anchor it into the pericardium.

23. The cardiac assist system of claim 15, wherein the partially elastic balloon comprises polyurethane.

24. The cardiac assist system of claim 15, wherein the partially elastic balloon has at least one fatigue property such that the partially elastic balloon is usable as anchored to the position anterior to the heart for at least one year without being exchanged.

25. The cardiac assist system of claim 15, wherein the partially elastic balloon is configured to partially yield to a pressure on the partially elastic balloon from the heart.

26. The cardiac assist system of claim 15, wherein the partially elastic balloon has an elastic modulus of from 4.7 MPa to 7.4 MPa.

27. A method for implanting a cardiac assist system, the method comprising:
(a) anchoring a pericardial anchor wire to a lateral aspect of a pericardium;
(b) advancing a guidewire lumen of an implantable cardiac assist catheter over an anchor wire into a position anterior to a heart; and
(c) anchoring a pneumatic effector to the position anterior to the heart, the pneumatic effector being coupled to a distal end of the implantable cardiac assist catheter and configured to be implanted beneath a pericardial sac of a subject and over a myocardial surface overlying a left ventricle of the subject, wherein the pneumatic effector comprises a partially elastic balloon;
(d) wherein the partially elastic balloon is configured to inflate with a cushioning effect on a myocardium of the subject at the position anterior to the heart in response to gas being driven thereto.

28. The method of claim 27, wherein the partially elastic balloon comprises polyurethane.

29. The method of claim 27, further comprising repeatedly expanding and contracting the partially elastic balloon anchored to the position anterior to the heart for at least one year without exchanging said partially elastic balloon, wherein the partially elastic balloon has at least one improved fatigue property such that said partially elastic balloon is usable for at least one year without being exchanged.

30. The method of claim 27, wherein the partially elastic balloon partially yields to a pressure on the partially elastic balloon from the heart.

31. The method of claim 27, wherein the partially elastic balloon has an elastic modulus of from 4.7 MPa to 7.4 MPa.

32. The method of claim 27, wherein anchoring the pericardial anchor wire comprises rotating and advancing a spiral anchor disposed on a distal end of the pericardial anchor wire into the pericardium.

33. A method for assisting cardiac function in a subject suffering from heart failure, the method comprising:
(a) measuring the subject's ECG to determine a cardiac rhythm; and
(b) delivering a driving gas to expand and contract a pneumatic effector implanted over the subject's left ventricle in response to the measured ECG, wherein the pneumatic effector comprises a partially elastic balloon;
(c) wherein the driving gas delivery is synchronized with the determined cardiac rhythm to cause the partially elastic balloon to compress a heart of the subject at a rate which matches the cardiac rhythm,
(d) wherein the partially elastic balloon is configured to inflate with a cushioning effect on a myocardium of the subject at a position anterior to the heart in response to gas being driven thereto.

34. The method of claim 33, wherein delivering driving gas comprises percutaneously accessing an implanted port connected to the pneumatic effector with a cannula through which the driving gas is delivered.

35. The method of claim 33, wherein the partially elastic balloon comprises polyurethane.

36. The method of claim 33, further comprising repeatedly expanding and contracting the partially elastic balloon implanted over the subject's left ventricle for at least one year without exchanging said partially elastic balloon, wherein the partially elastic balloon has at least one improved fatigue property such that said partially elastic balloon is usable used for at least one year without being exchanged.

37. The method of claim 33, wherein the partially elastic balloon partially yields to pressure on the partially elastic balloon from the heart.

38. The method of claim 33, wherein the partially elastic balloon has an elastic modulus of from 4.7 MPa to 7.4 MPa.

39. The method of claim 33, wherein the pneumatic effector is implanted beneath the patient's pericardial sac and over a myocardial surface.

40. The method of claim 33, wherein the ECG is measured with one or more electrodes located on an implanted pneumatic port.

41. The method of claim 33, wherein the ECG is measured with one or more electrodes located on the implanted pneumatic effector.

42. The method of claim 33, wherein the ECG is measured with a cannula acting as an ECG electrode.

43. The method of claim 33, wherein the ECG is measured with an external electrode.

44. The method of claim 33, wherein the ECG is measured with an ECG electrode located subcutaneously.

45. The method of claim 33, further comprising detecting an abnormal cardiac rhythm.

46. The method of claim 45, wherein delivery of the driving gas is stopped when an abnormal cardiac rhythm is detected.

47. The method of claim 45, wherein a rate of delivering the driving gas is changed when an abnormal cardiac rhythm is detected.

48. The method of claim 47, wherein the rate is below a rate of the detected cardiac rhythm.

49. The method of claim 47, wherein the rate is a predetermined fixed rate.

50. The method of claim 33, further comprising removing a cannula from an access site when an infection of the access site is observed.

51. The method of claim 50, further comprising treating the infection and replacing the cannula in an implanted port.

52. The method of claim 50, wherein the cannula is replaced in through a different site than enters a port through a different location.

53. The method of claim 52, wherein the cannula comprises a needle and the port comprise a needle-penetrable penetrable septum and the different location is a different region on the septum.

54. A cardiac assist system comprising:
(a) an external drive unit;
(b) an implantable cardiac assist catheter comprising a distal section;
(c) a pneumatic effector configured to be operatively coupled to the external drive unit, wherein the pneumatic effector is coupled to the distal section of the implantable cardiac assist catheter and configured to be implanted beneath a pericardial sac of a subject and over a myocardial surface overlying a left ventricle of the subject, and
(d) wherein the pneumatic effector comprises a balloon having a size of at least about 120 cc and configured to displace a left ventricular wall by at least about 4 cm.

55. The cardiac assist system of claim 54, wherein the distal section of the implantable cardiac assist catheter has a guidewire lumen with a first port proximal to the pneumatic effector and a second port distal to the pneumatic effector.

56. The cardiac assist system of claim 54, wherein the external drive unit comprises (a) a pump assembly; and (b) control circuitry configured to operate the pump assembly to actuate the pneumatic effector in response to the patient's sensed heart rhythm.

57. The cardiac assist system of claim 54, further comprising an implantable port configured to receive a percutaneously introduced cannula, wherein the implantable port is connected to supply a driving gas received from the percutaneously introduced cannula to the pneumatic effector.

58. The cardiac assist system of claim 57, further comprising, a connecting tube having a pump end attachable to a pump assembly and a cannula end attached to the percutaneously introduced cannula.

59. The cardiac assist system of claim 54, further comprising an anchor wire, wherein the anchor wire is configured to anchor the balloon to a position anterior to a heart.

60. The cardiac assist system of claim 59, wherein the anchor wire is configured to anchor the balloon to a position anterior to the heart.

61. The cardiac assist system of claim 60, wherein the anchor wire comprises a spiral anchor at a distal tip of the anchor wire.

62. The cardiac assist system of claim 61, wherein the spiral anchor is configured to be rotated and advanced into a pericardium of the subject to anchor it into the pericardium.

63. The cardiac assist system of claim 54, wherein the balloon is configured to generate a cardiac output of about 100 ml/min.

64. The cardiac assist system of claim 54, wherein the balloon is configured to displace a left ventricular wall by at least about 5 cm.

65. The cardiac assist system of claim 54, wherein the balloon is configured to generate a cardiac output approximately equivalent to 50% of a baseline cardiac output achieved via open heart massage at 60 bpm.

66. A method for implanting a cardiac assist system, the method comprising:
(a) anchoring a pericardial anchor wire to a lateral aspect of a pericardium;
(b) advancing an implantable cardiac assist catheter into a position anterior to a heart; and
(c) anchoring a pneumatic effector to the position anterior to the heart, the pneumatic effector being coupled to a distal end of the implantable cardiac assist catheter, wherein the pneumatic effector comprises a balloon having a size of at least about 120 cc;
(d) wherein a partially elastic balloon is configured to receive a driving gas delivered thereto to be inflated; and
(e) wherein the partially elastic balloon is configured to displace a left ventricular wall of the heart by at least about 4 cm in response to being inflated.

67. The method of claim 66, wherein the pneumatic effector is configured to be implanted beneath a pericardial sac of a subject and over a myocardial surface overlying a left ventricle of the subject.

68. The method of claim 66, wherein advancing the implantable cardiac assist catheter comprises advancing a guidewire lumen of an implantable cardiac assist catheter over the pericardial anchor wire into the position anterior to the heart.

69. The method of claim 66, wherein the balloon is configured to generate a cardiac output of about 100 ml/min.

70. The method of claim 66, wherein the balloon is configured to displace a left ventricular wall by at least about 5 cm.

71. The method of claim 66, wherein the balloon is configured to generate a cardiac output approximately equivalent to 50% of a baseline cardiac output achieved via open heart massage at 60 bpm.

72. The method of claim 66, wherein anchoring a pericardial anchor wire comprises advancing and rotating a spiral anchor disposed on a distal end of the pericardial anchor wire into the pericardium.

73. A method for assisting cardiac function in a subject suffering from heart failure, the method comprising:
(a) measuring the subject's ECG to determine a cardiac rhythm; and
(b) delivering a driving gas to expand and contract a pneumatic effector implanted over the subject's left ventricle in response to the measured ECG, wherein the pneumatic effector comprises a balloon having a size of at least about 120 cc;
(c) wherein the balloon is configured to displace a left ventricular wall by at least about 4 cm while expanding the balloon,
(d) wherein the driving gas delivery is synchronized with the determined cardiac rhythm to cause the balloon to compress a heart of the subject at a rate which matches the cardiac rhythm.

74. The method of claim 73, wherein delivering driving gas comprises percutaneously accessing an implanted port connected to the pneumatic effector with a cannula through which the driving gas is delivered.

75. The method of claim 73, wherein the pneumatic effector is implanted beneath the patient's pericardial sac and over a myocardial surface.

76. The method of claim 73, wherein the ECG is measured with one or more electrodes located on an implanted pneumatic port.

77. The method of claim 73, wherein the ECG is measured with one or more electrodes located on the implanted pneumatic effector.

78. The method of claim 73, wherein the ECG is measured with a cannula acting as an ECG electrode.

79. The method of claim 73, wherein the ECG is measured with an external electrode.

80. The method of claim 73, wherein the ECG is measured with an ECG electrode located subcutaneously.

81. The method of claims 73, further comprising detecting an abnormal cardiac rhythm.

82. The method of claim 81, wherein delivery of the driving gas is stopped when an abnormal cardiac rhythm is detected.

83. The method of claim 81, wherein a rate of delivering the driving gas is changed when an abnormal cardiac rhythm is detected.

84. The method of claim 83, wherein the rate is below a rate of the detected cardiac rhythm.

85. The method of claim 83, wherein the rate is a predetermined fixed rate.

86. The method of claim 73, further comprising removing a cannula from an access site when an infection of the access site is observed.

87. The method of claim 86, further comprising treating the infection and replacing the cannula in an implanted port.

88. The method of claim 87, wherein the cannula is replaced in through a different site than enters the implanted port through a different location.

89. The method of claim 88, wherein the cannula comprises a needle and the implanted port comprise a needle-penetrable penetrable septum and the different location is a different region on the septum.

90. The method of claim 73, wherein the balloon is configured to generate a cardiac output of about 100 ml/min.

91. The method of claim 73, wherein the balloon is configured to displace a left ventricular wall by at least about 5 cm.

92. The method of claim 73, wherein the balloon is configured to generate a cardiac output approximately equivalent to 50% of a baseline cardiac output achieved via open heart massage at 60 bpm.

93. An implantable cardiac assist system, comprising:
(a) an external drive unit;
(b) an anchor wire configured to be anchored to a pericardium of a subject;
(c) an implantable cardiac assist catheter comprising a distal section; and
(d) a pneumatic effector configured to be operatively coupled to the external drive unit, wherein the pneumatic effector is coupled to the distal section of the implantable cardiac assist catheter and configured to be implanted beneath a pericardial sac of the subject and over a myocardial surface overlying a left ventricle of the subject,
(e) wherein the implantable cardiac assist catheter is configured to be advanced over the anchor wire via a guidewire lumen, and
(f) wherein one or more of
(i) the pneumatic effector comprises a balloon having a size of at least about 120 cc, wherein the balloon is configured to displace a left ventricular wall by at least about 4 cm while expanding the balloon;
(ii) the pneumatic effector comprises a partially elastic balloon configured to inflate with a cushioning effect on a myocardium of the subject at a position anterior to the heart in response to gas being driven thereto; or
(iii) the distal section of the implantable cardiac assist catheter has a guidewire lumen with a first port proximal to the pneumatic effector and a second port distal to the pneumatic effector.

94. A method for implanting a cardiac assist system, the method comprising:
(a) anchoring a pericardial anchor wire to a lateral aspect of a pericardium;
(b) advancing a guidewire lumen of an implantable cardiac assist catheter over the anchor wire into a position anterior to a heart; and
(c) anchoring a pneumatic effector to the position anterior to the heart, the pneumatic effector being coupled to a distal end of the implantable cardiac assist catheter and configured to be implanted beneath a pericardial sac of the subject and over a myocardial surface overlying a left ventricle of the subject;
(d) wherein one or more of
(i) the pneumatic effector comprises a balloon having a size of at least about 120 cc, wherein the balloon is configured to displace a left ventricular wall by at least about 4 cm while expanding the balloon;
(ii) the pneumatic effector comprises a partially elastic balloon configured to inflate with a cushioning effect on a myocardium of the subject at a position anterior to the heart in response to gas being driven thereto; or
(iii) a distal section of the implantable cardiac assist catheter has a guidewire lumen with a first port proximal to the pneumatic effector and a second port distal to the pneumatic effector.
